(19) United States
(12) Patent Application Publication
(10) Pub. No.: US 2026/0108882 A1
(43) Pub. Date: Apr. 23, 2026

(54) Addressable Microfluidic Systems for Controlled Fluid Flow, Diffusion, and Micro-Organism Behavior

(71) Applicant: New Jersey Institute of Technology, Newark, NJ (US)

(72) Inventors: Roman Voronov, Oklahoma City, OK (US); Sergei Chapek, Austin, TX (US); Huu Anh Minh Nguyen, Newark, NJ (US)

(73) Assignee: New Jersey Institute of Technology, Newark, NJ (US)

(21) Appl. No.: 19/309,238

(22) Filed: Aug. 25, 2025

Related U.S. Application Data
(60) Provisional application No. 63/710,785, filed on Oct. 23, 2024.

(51) Int. Cl. B01L 3/00; C12M 3/06

(52) U.S. Cl. CPC B01L 3/502738; C12M 23/16; B01L 2200/028; B01L 2400/0622

(57) ABSTRACT

An addressable microfluidics device is provided. The device includes a chamber, a first flow channel pair including first and second flow channels fluidly connected to sides of the chamber, and a second flow channel pair including a third flow channel and a fourth flow channel fluidly connected to sides of the chamber. The device includes at least one bypass flow channel connecting either the first flow channel and the second flow channel, or the third flow channel and the fourth flow channel, to bypass the chamber. The device includes a first side valve associated with either the first flow channel or the third flow channel, and a second side valve associated with either the second flow channel or the fourth flow channel. The first and second side valves are selectively operable into open or closed positions to divert flow through the at least one bypass flow channel.

### CROSS-REFERENCE TO RELATED APPLICATIONS

This application claims the benefit of a co-pending, commonly assigned U.S. Provisional Patent Application No. 63/710,785 , which was filed on Oct. 23, 2024. The entire content of the foregoing provisional application is incorporated herein by reference.

### STATEMENT OF GOVERNMENT SUPPORT

This invention was made with government support under Grant No. 2141029 awarded by the National Science Foundation (NSF), and Grant No. GM145610 awarded by the National Institutes of Health (NIH). The government has certain rights in the invention.

### FIELD OF THE DISCLOSURE

The present disclosure relates to a multiwall addressable microfluidics device and, in particular, to a high-throughput microfluidics device usable for chemical and biological assays for streamlining and enhancing the process of conducting a large number of experiments or tests simultaneously, providing a versatile addressable microfluidic system for controlled fluid flow, diffusion, cell migration across one-dimensional, two-dimensional and three-dimensional configurations, tissue patterning, engineered tissue scaffolds, culture scaffolds, and micro-organism behavior (e.g., cells, yeast, bacteria, or the like).

### BACKGROUND

Microfluidics devices are generally used to perform experiments using chemical and biological assays. As the number of parameters and/or experiments increases, the traditional microfluidics devices have been modified to accommodate the higher demand for testing wells. This higher demand results in complications due to the larger number of flow channels and the architecture for regulating flow to these channels. For example, the internal plumbing of the traditional devices can necessitate that each separate well or chamber (from here on referred to as “addresses”) be operated by its own individual plumbing component to route fluids and samples to and from the address. However, this can be a significant challenge because the plumbing should be both scalable to high density devices (i.e., number of chambers per volume should be as high as possible) and, at the same time, be capable of maintaining a high density targeting throughout its pore space. This scaling problem is illustrated using a two-dimensional example in FIG. 1.

In particular, FIG. 1 shows a traditional microfluidics device 10 in a 3×3 grid format with inefficient X*Y scaling, and resolution limitations due to crowding of supply channels. It should be understood that the 3×3 grid is shown for example purposes only, and any grid size could be used. For example purposes, a 3×3 grid of microfluidic wells (addresses 12) is actuated via a separate channel 14 dedicated to each of the addresses 12. The device 10 of FIG. 1 suffers from poor scalability and crowding. With respect to poor scaling, the number of flow channels 14 required to actuate each individual address 12 in the grid scales as X*Y, which is the worst-case scenario. Thus, for each additional address 12, a new flow channel 14 must be added.

With respect to crowding (e.g., areas 16), the spacing available for the channels 14 is limited by the separation distance between the neighboring columns of addresses 12 (which should be as small as possible in order to be able to manipulate and analyze the culture with a single cell spatial resolution). There is a concept in the microfluidics industry that solves this problem by including orthogonally-blocking channels 22 in the microfluidics device 20 of FIG. 2. (See, e.g., Wang, H. Y., et. al., A microfluidic cell array with individually addressable culture chambers. Biosens Bioelectron, 24(4): p. 613-7 (2008); Leung, K., et al., A programmable droplet-based microfluidic device applied to multiparameter analysis of single microbes and microbial communities. Proc Natl Acad Sci U S A, 109(20): p. 7665-70 (2012); and Gao, Y., et al., Digital microfluidic programmable stencil (DMPS) for protein and cell patterning. RSC Advances, 6(104): p. 101760-101769 (2016)).

The device 20 of FIG. 2 includes efficient (X+Y) scaling and the resolution is not limited by crowding. In particular, the device 20 includes addresses 24 with flow channels 26 feeding groups of addresses 24 (e.g., three addresses 22 per flow channel 26), which are intersected at intersections 28 of blocking channels 22. The intersections 28 occur between addresses 24 to allow for controlled regulation of flow to the respective addresses 24. By using this combination of flow and blocking channels 26, 22 (e.g., valve actuating), (X+Y) scaling is achieved (i.e., only three flow and only three blocking channels are required, as opposed to a total of nine dedicated flow channels in the X*Y scaling of FIG. 1). Thus, the orthogonal plumbing, such as in the device 20 of FIG. 2, yields the best possible scaling and the address density is no longer limited by crowding.

Although addressable microfluidics chips with over 1000 addresses have been previously made in the industry, there are different plumbing approaches (each of which has its own trade-offs) to achieving independent control and manipulation over the experimentation wells. (See, e.g., Thorsen, T., et al., Microfluidic large-scale integration. Science, 298(5593): p. 580-4 (2002); Melin, J., et al., Microfluidic large-scale integration: the evolution of design rules for biological automation. Annu Rev Biophys Biomol Struct, 36: p.213-31 (2007); Mark, D., et al., Microfluidic lab-on-a-chip platforms: requirements, characteristics and applications. Chem Soc Rev, 39(3): p. 1153-82 (2010); and Araci, I. E., et al., Recent developments in microfluidic large scale integration. Curr Opin Biotechnol, 25: p. 60-8 (2014)). For example, FIG. 3 shows one traditional configuration of an addressable array 30 of an arbitrarily-chosen 4×4 size for a simple demonstration (though the actual grid size is a free parameter). (See, e.g., Wang, H. Y., et. al., A microfluidic cell array with individually addressable culture chambers. Biosens Bioelectron, 24(4): p. 613-7 (2008); Leung, K., et al., A programmable droplet-based microfluidic device applied to multiparameter analysis of single microbes and microbial communities. Proc Natl Acad Sci U S A, 109(20): p. 7665-70 (2012); Gao, Y., et al., Digital microfluidic programmable stencil (DMPS) for protein and cell patterning. RSC Advances, 6(104): p. 101760-101769 (2016)). In FIG. 3, each of the addresses 32 in the array 30 are shown with letters denoting different payload (e.g., cells and/or chemicals) contents of the wells, which are surrounded by O-shaped pneumatic valves 34. Control channels 40 are used to regulate opening and closing of the valves 34. The inset at the bottom of FIG. 3 acts as a legend, showing valve positions and flows for use in the discussion of the main portion of FIG. 3. When the valve 34 is “closed” (represented by a solid circle around the address 32), the fluid traveling through the channels 36 is re-routed around the address 32 via a thin bypass channel 38. However, when a valve 34′ is “open” (represented by a hollow circle around the address 32), the corresponding address 32 can either deliver or withdraw (depending on the direction of the flow in the channels 36) the fluid carrying a chemical and/or cellular payload to/from the well. The arrows in the closed and open valve diagrams represent the direction of flow, which assumes a payload delivery to (as opposed to sampling at) an address 32. Hence, the presence of the bypass channel 38 enables routing of the payloads around some addresses 32 and not the other addresses 32 in the same row of the matrix. This provides one approach to achieving addressability in high-throughput microfluidics devices.

In the plumbing configuration of FIG. 3, payload exchange can only be achieved between different addresses 32 located in the same row (e.g., between the A, B, C and D wells along the same channel 36). It is not possible to send the payloads directly from one row of addresses 32 to another (e.g., from A to E in a column-wise manner) without traversing the entire device or array 30 first. For this reason, the plumbing layout in FIG. 3 is termed as a “Row-wise Addressable Plumbing Layout with Distinct Bypasses”. This type of plumbing layout can be a limitation in case cellular and/or chemical exchange is desired to occur between neighboring addresses 32 in the column-wise (i.e., orthogonal) direction. For example, in combinatorial screening studies where reaction components from different rows of addresses need to be mixed with each other at some point during the experiment.

To resolve this limitation, FIG. 4 shows a plumbing layout array 50 that is capable of sending payloads in both the horizontal and the orthogonal directions. (See, e.g., Hansen, C. L., et al., High throughput screening materials crystallization of materials, USPTO, Editor. (2000)). However, this is accomplished by sacrificing every other column 52, 54 of addresses 56 to effectively serve as bypass channels instead. The array 50 includes multiple columns of addresses 56 (such as columns 52, 54), and intersecting rows of addresses 56 (such as row 58). The capital letters at the addresses 56 indicate addresses 56 that contain payloads, while those without capital letters are blanks used essentially as part of a bypass channel. The columns 52, 54 and rows 58 define flow channels, while channels 60 are blocking channels that regulate valve 62 actuation to open and close the respective flow channels. Valves 62 can be interconnected by additional intermediate channels 64 in series, with further channels 66 used to connect the valves 62 to the main blocking channel 60.

In other words, unlike in the configuration of FIG. 3, the configuration of FIG. 4 does not have distinct bypass channels. Instead, entire columns 52, 54 of addresses 56 are used for bypassing. Specifically, the payload addresses 56 in FIG. 4 are labeled with letters (e.g., A, B, C, D), while the columns 52, 54 in-between them are essentially blank when they are not used. This pattern is repeated in an alternating manner for every other column. If payload C needed to be moved orthogonally upward, flow would be applied to the row 58 containing payload C and payload G, such that both payloads would be shifted one grid point to the right. Then, valve 1 at the bottom of column 52 would be actuated to open and apply pressure in the upward direction to move payload C to a different row. Meanwhile, valve 2 at the bottom of column 54 would remain closed to not disturb payload G, which would be stored in a temporary position. Subsequently, after payload C has been moved out of the way, payload G could be returned to its initial position by reversing the flow direction in the row 58. Finally, payload C could be placed into any of the positions (e.g., A, B, E or F) in one of higher rows 58 by performing similar manipulations. Although more capable than the “Row-Wise Addressable Plumbing Layout With Distinct Bypasses” design, the “Row-Column Addressable Plumbing Layout With Sacrificial Bypass Columns” approach wastes about 50% of precious device space that could otherwise be used for performing more high throughput experiments. In particular, as illustrated in FIG. 4, only half of the wells in the 4×4 addressable grid are filled with letters in this configuration relative to the plumbing configuration of FIG. 3 due to the necessity of columns being used for bypass channel flow.

Furthermore, the payloads in the FIG. 4 configuration cannot be moved in the horizontal direction without moving their row 58 neighbors out of the way. For example, it is not possible to move payload D to the right without disturbing payload H. First, both payloads D, H would need to be moved to the right; then payload H would be raised by one row; and finally, payload D could be moved further to the right. Likewise, a payload that is being relocated cannot simply move orthogonally through its own column 51, 53. Instead, it has to go around through the bypass channel (as was the case in the payload C example above). Unfortunately, this results in an inefficient process, because the payloads must travel over larger distances and longer times (during which they can disperse or become cross-contaminated).

Therefore, an improved plumbing layout is needed for an addressable microfluidics device that can move payloads in both directions, and without sacrificing channels or disturbing the neighboring payloads.

### SUMMARY

In accordance with embodiments of the present disclosure, an exemplary addressable microfluidics device is provided. It should be understood that the term “device” refers to a structure having a single chamber, and multiple such devices can be interconnected structurally and fluidly to form a “system”. The device includes a chamber, a first flow channel pair including a first flow channel and a second flow channel fluidly connected to sides of the chamber, and a second flow channel pair including a third flow channel and a fourth flow channel fluidly connected to sides of the chamber. The device includes at least one bypass flow channel connecting either (i) the first flow channel and the second flow channel, or (ii) the third flow channel and the fourth flow channel, to bypass the chamber. The device includes a first side valve associated with either the first flow channel or the third flow channel, and a second side valve associated with either the second flow channel or the fourth flow channel, wherein the first and second side valves are selectively operable into open or closed positions to divert flow through the at least one bypass flow channel.

The chamber can be capable of (i) receiving and/or sending a fluid (ii) receiving and/or sending the fluid with a sample, (iii) receiving and/or sending the fluid with a chemical signal, and/or (ii) hosting a reaction and/or a culture. In some embodiments, the first and second flow channels can extend along a first plane (e.g., the same plane). In some embodiments, the first and second flow channels can extend on different planes relative to each other. In some embodiments, the third and fourth flow channels can extend along a second plane offset from the first plane. In some embodiments, the third and fourth flow channels can extend on different planes relative to each other. In some embodiments, each of the first, second, third and fourth flow channels can extend on different planes relative to each other. In embodiments where the first and second flow channels extend along the first plane and the third and fourth flow channels extend along the second plane, the second plane can offset from the first plane by about 90°.

In some embodiments, the at least one bypass flow channel can include a first bypass flow channel connecting the first flow channel and the second flow channel, and a second bypass flow channel connecting the third flow channel and the fourth flow channel, to bypass the chamber. The first side valve can be associated with the first flow channel and the second side valve can be associated with the second flow channel. The first and second side valves can be selectively operable into the open or closed positions to divert flow through the first bypass flow channel. The device can include a third side valve associated with the third flow channel and a fourth side valve associated with the fourth flow channel. The third and fourth side valves can be selectively operable into open and closed positions to divert flow through the second bypass flow channel.

In some embodiments, the first side valve and the second side valve can operate concurrently to open or close in a joined manner. In some embodiments, the first side valve and the second side valve can be independently operable relative to each other to open and close.

In some embodiments, the device can include at least one bypass valve associated with the at least one bypass flow channel. The at least one bypass valve can be operable into open or closed positions to divert flow through the at least one bypass flow channel. In some embodiments, the device can include a fifth flow channel and a sixth flow channel fluidly connected to sides of the chamber. The first and second flow channels can extend along a first plane, the third and flow channels can extend along a second plane offset from the first plane, and the fifth and sixth flow channels can extend along a third plane offset from both the first plane and the second plane. In some embodiments, each of the flow channels can extend along different planes from the other flow channels. The at least one bypass flow channel can include a third bypass flow channel connecting the fifth flow channel and the sixth flow channel. The device can include a fifth side valve associated with the fifth flow channel and a sixth side valve associated with the sixth flow channel. The fifth and sixth side valves can be selectively operable into open or closed positions to divert flow through the third bypass flow channel.

In accordance with embodiments of the present disclosure, an exemplary method for microfluidics operation is provided. The method includes passing a fluid through at least one of (i) a first flow channel fluidly connected to a chamber of an addressable microfluidics device, (ii) a second flow channel fluidly connected to the chamber, (iii) a third flow channel fluidly connected to the chamber, (iv) or a fourth flow channel fluidly connected to the chamber. The first flow channel and the second flow channel form a first flow channel pair connected to sides of the chamber, and a third flow channel and a fourth flow channel form a second flow channel pair connected to sides of the chamber. The method includes selectively operating a first side valve associated with either the first flow channel or the third flow channel, and/or selectively operating a second side valve associated with either the second flow channel or the fourth flow channel, into open or closed positions to divert flow through at least one bypass flow channel connecting either (i) the first flow channel and the second flow channel, or (ii) the third flow channel and the fourth flow channel, to bypass the chamber.

In accordance with embodiments of the present disclosure, an exemplary addressable microfluidics device is provided. The device includes a chamber, a first flow channel fluidly connected to a first side of the chamber, a second flow channel fluidly connected to a second side of the chamber, and a bypass flow channel connecting the first flow channel and the second flow channel. The device includes a first side valve associated with the first flow channel, and a second side valve associated with the second flow channel. The first and second side valves are selectively operable into open and closed positions to divert flow through the bypass flow channel.

The chamber can be capable of (i) receiving and/or sending a fluid (ii) receiving and/or sending the fluid with a sample, (iii) receiving and/or sending the fluid with a chemical signal, and/or (ii) hosting a reaction and/or a culture. In some embodiments, the first side valve and the second side valve can be independently operable relative to each other to open and close. The device can include at least one bypass valve associated with the bypass flow channel. The at least one bypass valve can be operable into open or closed positions to divert flow through the bypass flow channel.

In accordance with embodiments of the present disclosure, various microfluidics devices/systems are provided. The microfluidic systems are capable of selectively controlling fluid flow, diffusion, cell migration, and/or micro-organism behavior across various dimensional configurations, including one dimensional (1D), two dimensional (2D), and three dimensional (3D) individually-addressable arrays of wells. As discussed herein, 1D refers to fluid flow along a single direction between two flow channels, 2D refers to fluid flow in two directions between four flow channels, and 3D refers to fluid flow in three directions between six flow channels. The microfluidic systems discussed herein can be used for running and studying various reactions as well.

The device utilizes a series of valves or actuators managed by an integrated controller (e.g., a system of electronics driven by a computer), enabling precise control over fluidic connections between adjacent wells. In its 1D configuration, the system facilitates diffusive communication and/or cell migration along linear arrays of wells. The 2D and 3D configurations can operate with or without diffusive communication and/or cell migration, offering higher experimental throughput and flexibility in both setup and applications. The device is adaptable to different operational modes, including single-plane or single-volume modes, high-throughput screening, and subdivided sub-plane or sub-volume modes. These features make the system highly versatile for a wide range of applications, such as (but not limited to) chemical reactions, biological assays, cell cultures, targeted delivery of fluids or cells, and dynamic manipulation of cell behavior or tissue patterning. The devices include various valve types and plumbing modifications to optimize performance and address fabrication challenges, ensuring robustness and broad applicability in research and clinical environments.

In some embodiments, the device can be a 1D addressable microfluidic system with diffusive communication and cell migration between adjacent wells in the absence of flow. The device includes one or more linear arrays of addressable wells. Each array is arranged in a single dimension (1D). The term “addressable” refers to the capability of selectively controlling and directing fluid flow, diffusion, and cell migration between specific wells through the use of valves or actuators. The device includes a set of valves or actuators, controlled by a controller, enabling the selective control of fluidic connections between adjacent wells within each linear array along the 1D direction. The term “controller” refers to an integrated system or component responsible for managing the operation of said valves or actuators. The device includes means for allowing diffusive communication and cell migration between adjacent wells within each linear array in the absence of active fluid flow. Diffusion and/or cell migration occur through selectively openable channels connecting said wells within the same linear array. The linear arrays, if multiple, are joined before and after the addressable matrix of wells, but fluids, cells, or other materials within the arrays are confined to travel only along the 1D direction of each array, with no possibility of transfer in the direction orthogonal to the linear arrays.

The wells are configured to hold chemical, biological samples, or cells, and allow for the passive exchange of molecules and cell migration between adjacent wells via diffusion and movement, restricted to the 1D direction of each linear array. The device can include a controller configured to open or close the channels between adjacent wells within each linear array, thereby controlling the onset and duration of diffusive communication and cell migration along the 1D direction.

In some embodiments, the device can be a 2D addressable microfluidic system without diffusive communication or cell migration between adjacent wells. The device includes a single or multiple 2D addressable arrays of wells, each array arranged in two dimensions (2D). The term “addressable” refers to the capability of selectively controlling and directing fluid flow between specific wells through the use of valves or actuators. The device includes a set of valves or actuators, controlled by a controller, enabling the selective control of fluidic connections between adjacent wells within each 2D array in both horizontal and vertical directions. The term “controller” refers to an integrated system or component responsible for managing the operation of said valves or actuators. Fluid movement is restricted to the 2D plane of each array, with no possibility of fluid transfer in the third direction (even if the 2D arrays are stacked in 3D) unless the fluid travels before entering or after leaving the addressable matrix of wells. the controller is configured to manage the operation of the valves or actuators, directing fluid flow within the 2D plane of each array and ensuring that fluid movement is confined to preselected channels.

In some embodiments, the device can be a 2D addressable microfluidic system with diffusive communication and cell migration between adjacent wells in the absence of flow. The device includes a single or multiple 2D addressable arrays of wells, each array arranged in two dimensions (2D). The term “addressable” refers to the capability of selectively controlling and directing fluid flow, diffusion, and cell migration between specific wells through the use of valves or actuators. The 2D addressable arrays may be stacked in 3D. The device includes a set of valves or actuators, controlled by a controller, enabling the selective control of fluidic connections between adjacent wells within each 2D array in both horizontal and vertical directions. The term “controller” refers to an integrated system or component responsible for managing the operation of said valves or actuators. The device includes means for allowing diffusive communication and cell migration between adjacent wells within each 2D array in the absence of active fluid flow. Diffusion and cell migration occur through selectively openable channels connecting said wells in both horizontal and vertical directions within the same 2D array. Fluid and cell movement is restricted to the 2D plane of each array, with no possibility of transfer in the third direction unless the fluid or cells travel before entering or after leaving the addressable matrix of wells.

The wells are configured to hold chemical, biological samples, or cells, and allow for the passive exchange of molecules and cell migration between adjacent wells via diffusion and movement, restricted to the 2D plane of each array. The device includes a controller configured to selectively enable or disable diffusive communication and cell migration between wells, allowing for dynamic control of diffusion and migration pathways within each 2D array.

In some embodiments, the device can be a 3D addressable microfluidic system without diffusive communication or cell migration between adjacent wells. The device includes a three-dimensional (3D) array of addressable wells. The term “addressable” refers to the capability of selectively controlling and directing fluid flow between specific wells through the use of valves or actuators. The device includes a set of valves or actuators, controlled by a controller, enabling the selective control of fluidic connections between adjacent wells within the 3D array. The term “controller” refers to an integrated system or component responsible for managing the operation of said valves or actuators. The device includes means for controlling fluidic flow between adjacent wells in all three dimensions, excluding diffusive communication and cell migration, to facilitate directed fluid movement through the array.

The wells are configured to hold chemical or biological samples and allow for the directed movement of fluids between adjacent wells without permitting diffusion-based communication or cell migration. The device includes a controller to manage the flow pathways within the 3D array, ensuring that fluid movement is confined to preselected channels.

In some embodiments, the device can be a 3D addressable microfluidic system with diffusive communication and cell migration between adjacent wells in the absence of flow. The device includes a three-dimensional (3D) array of addressable wells. The term “addressable” refers to the capability of selectively controlling and directing fluid flow, diffusion, and cell migration between specific wells through the use of valves or actuators. The device includes a set of valves or actuators, controlled by a controller, enabling the selective control of fluidic connections between adjacent wells within the 3D array. The term “controller” refers to an integrated system or component responsible for managing the operation of said valves or actuators. The device includes means for allowing diffusive communication and cell migration between adjacent wells in all three dimensions in the absence of active fluid flow. Diffusion and cell migration occur through selectively openable channels connecting said wells.

The wells are configured to hold chemical, biological samples, or cells, and allow for the passive exchange of molecules and cell migration between adjacent wells via diffusion and movement in all three spatial dimensions. The device includes a controller configured to dynamically control the diffusive communication and cell migration pathways between wells, enabling or disabling diffusion and migration in any combination of the three dimensions.

In some embodiments, various operational modes for 2D and 3D addressable systems are provided. Means for configuring the device to operate in different modes includes the following. A single 2D plane mode where all wells within the plane are interconnected, enabling continuous manipulation across the entire plane (for 2D arrays). A single 3D volume mode where all wells are interconnected in three dimensions, enabling continuous manipulation throughout the entire volume (for 3D arrays). A high-throughput screening mode where wells within the 2D or 3D array are isolated from each other, allowing independent experiments in each well. A 2D sub-plane mode or 3D sub-volume mode where the device is divided into multiple 2D sub-planes or 3D sub-volumes, each including interconnected wells that are isolated from other sub-planes or sub-volumes, enabling multiple independent experiments within the same device.

The controller is configured to dynamically switch between these operational modes, allowing for real-time reconfiguration of the device and the conduct of time-dependent experiments. Various valve type and plumbing modifications can be provided. The type of valve utilized in the microfluidic plumbing to block flow channels and bypasses can be selected from any suitable valve type known in the art, with specific valve types being exemplary and not limited to those mentioned, based on convenience, fabrication considerations, and experimental requirements. The valve type and associated plumbing design can be modified to overcome geometric constraints and fabrication limitations, including but not limited to the introduction of reinforcements to prevent delamination in vertically-oriented membranes, adjustments to layer thickness or crosslinking times near valves to enhance structural integrity, and plumbing modifications such as U-turns in vertical channels to facilitate horizontal squeezing of vertical flow channels. The device can include modifications to the valve geometry, including the addition of “doormat” elements to narrow channel profiles and improve valve function. Bypass channels can include straight horizontal and vertical segments with right-angle turns to simplify fabrication, avoiding diagonal and curved features that may pose challenges in layer-by-layer 3D printing or other fabrication methods, although other configurations are also envisioned.

The device can be configured to host and support chemical reactions, cell cultures, or biological assays within the addressable wells. The device can be used for the targeted delivery of fluids, chemicals, drugs, nutrients, metabolites, factors, digestion enzymes, or cells to specific wells or regions within the device. The device can be used for targeted probing, sampling, or analysis of fluids, chemicals, or cells within specific wells or regions, including flowing samples out for ex-situ analysis. The device can be configured for waste removal, modulating cell culture development, controlling cell behavior, patterning tissues, or other related biological applications. In some embodiments, one or more of the chambers of the device can be loaded with gel, mesh or a similar material to prevent cell migration between adjacent chambers, thereby allowing certain chambers to be bypassed.

It should be understood that various components and/or configurations of the devices discussed herein can be changed to maximize the space between components for operation and/or fabrication. Thus, flow channels, bypass channels, valves, or combinations thereof, can have different orientations and/or configurations. The size and shape of the components could also be varied. For example, if a 3D printer is used, the diameter of certain channels (e.g., vertical vs horizontal) can be increased to compensate for fabrication limitations. Flow channels are illustrated as being substantially linear for clarity, but could be configured to have non-straight paths with detours. The flow channels and bypass channels can define different cross-sectional profiles, e.g., circular, triangular, oval, hexagonal, square, rectangular, or the like.

The culture chamber can also have a varying shape/size, e.g., cube, cylinder, or the like. In some embodiments, the angle of the bypass channel elbow can be changed or different channel/bypass shapes and pathways can be used. In some instances, rows or columns may not have a cell chamber with valves (e.g., sacrificial columns), or can have more than one culture chamber between a single bypass flow channel. In some instances, spacing between different elements can be varied or be non-uniform to accommodate spacing and fabrication requirements. In some embodiments, connections between flow channels and valves can be offset, e.g., connecting to the top of the valve, connecting to the middle of the valve, connecting to the bottom valve, or the like. Although the devices are illustrated with neighboring bypass channels overlapping or connecting with each other for compactness, in some embodiments, the bypass channels can be separated from each other (with the understanding that such design change would lead to a lower density of experiments per system).

In some embodiments, various other microfluidic elements can be inserted into the devices. For example, manifolds, mixers, flush channels, pumps, multiplexors, sieves, or the like, could be used. Microsieves can be on-chip filters designed to separate particles or cells. Hydrodynamic filtration structures can use channel geometry to divert particles by size, such as pillar arrays. Membrane barriers can be thin, permeable barriers integrated into channels for selective filtration.

Various flow control elements can also be used, e.g., on-chip valves, micropumps,, or the like. In terms of on-chip valves, pneumatic microvalves can be soft lithography-based valves that deform elastomeric layers using pneumatic actuation, quake valves can be elastomeric valves used in PDMS multilayer devices, check valves can be microfluidic structures that allow flow in only one direction, and thermal valves can be valves actuated by localizing heating to control flow. In terms of micropumps, peristaltic micropumps can be sequentially actuated valves to create a peristaltic motion for fluid pumping, electrokinetic pumps can generate flow using electroosmosis directly within the chip, and capillary pumps can use surface tension and channel geometry for passive flow control.

Bubble management elements can also be used, e.g., on-chip bubble traps, mixing elements, or the like. On-chip bubble traps can have special geometries or chambers that capture bubbles, preventing them from entering sensitive areas. On-chip bubble traps can include degassing channels, which are thin, gas-permeable PDMS channels to remove dissolved gases and avoid bubble formation. Passive micromixers can include serpentine channels with winding channels that enhance mixing through diffusion and advection, and herringbone mixers with grooved structures that create chaotic advection for effective mixing. Active micromixers can use electric fields (e.g., AC electroosmosis) or magnetic forces to enhance mixing within the microchannel.

Droplet generation elements can also be used, e.g., T-junctions, which are on-chip structures where two channels intersect perpendicularly to create uniform droplets. T-junctions can include flow-focusing devices which are co-flowing streams of immiscible fluids that generate droplets, or electrowetting arrays including on-chip electrodes that modulate droplet size by changing surface tension.

Separation elements can also be used, e.g., micropillar arrays, which are fixed arrays used to sort particles or cells based on size, shape, or deformability. Micropillar arrays can include dielectrophoretic traps that include on-chip electrodes generating non-uniform electric fields to manipulate particles and cells, or inertial separation channels that use inertial effects in curved channels to separate particles based on size.

Detection and analysis elements can also be used, e.g., optical detection windows, which are transparent regions on the chip for spectrophotometric or fluorescence analysis. Optical detection windows can include electrochemical sensors that are on-chip electrodes for real-time chemical analysis, or conductivity channels that re channels with embedded electrodes for measuring changes in conductivity.

Droplet and bubble manipulation elements can also be used, e.g., droplet splits, which are structures that passively split larger droplets into smaller volumes. Droplet splitters can include droplet mergers that have on-chip designs that merge two or more droplets through channel geometry or electric fields, or bubble coalescers that are channels designed to guide smaller bubble to coalesce into larger ones, facilitating removal.

Temperature control elements can also be used, e.g., microheaters, which are integrated resistive heaters for controlling localized temperature in microchannels. Microheaters can include thermoelectric Peltier units that are small elements integrated on-chip for temperature modulation.

Capillary elements can also be used, e.g., capillary valves that are structures with defined channel geometry to exploit capillary forces, preventing or permitting flow based on design, or capillary pumps that are designed channel paths that drive fluid using capillary action to achieve passive flow control.

Flow resistance and dividers can also be used, e.g., flow resistors, that are on-chip constructions that create controller resistance to adjust flow rates. Flow resistors can include flow splitters, which are junctions that divide a single flow path into multiple paths for parallel fluid manipulation.

Cell culture and capture elements can also be used, e.g. microwells, that are small-scale culture chambers integrated on-chip for individual cells or colonies. Microwells can include cell capture traps that have specialized geometries to isolate single cells, often combined with hydrodynamic forces or DEP.

Surface modification elements can also be used, e.g., patterned hydrophobic/hydrophilic regions, that modify specific areas to control droplet movement and merging. Such regions can include chemical coatings that functionalize channel surfaces for specific interactions, such as antibody-antigen binding or anti-fouling. Various sensors and/or on-chip electronic elements could also be integrated into the devices/systems.

Any combination and/or permutation of the embodiments is envisioned. Other objects and features will become apparent from the following detailed description considered in conjunction with the accompanying drawings. It is to be understood, however, that the drawings are designed as an illustration only and not as a definition of the limits of the present disclosure.

### BRIEF DESCRIPTION OF THE DRAWINGS

To assist those of skill in the art in making and using the exemplary multiwall addressable microfluidics devices, reference is made to the accompanying figures, wherein:

FIG. 1 is a diagrammatic view of a traditional naïve microfluidics device with inefficient X*Y scaling and resolution limitations due to crowding of supply channels.

FIG. 2 is a diagrammatic view of a traditional addressable microfluidics device with efficient X+Y scaling and resolution not limited by crowding.

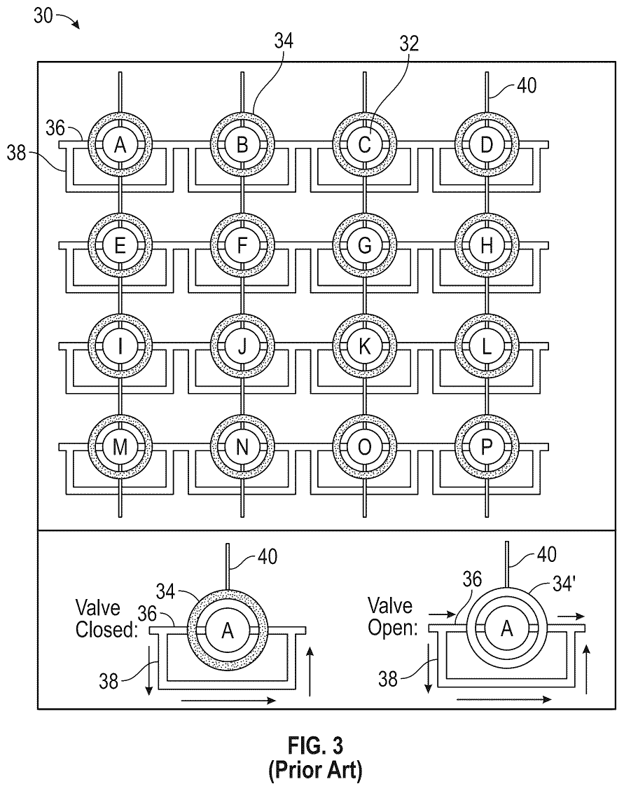

FIG. 3 is a diagrammatic view of a traditional microfluidics device with a row-wise addressable plumbing layout with distinct bypasses.

FIG. 4 is a diagrammatic view of a traditional microfluidics device with a row-column addressable plumbing layout in which every other column is sacrificed to serve as a bypass channel.

FIG. 5 is a diagrammatic view of an exemplary 2D microfluidics device including a row-column addressable plumbing layout with distinct bypass channels to define a unit module.

FIG. 6 is a diagrammatic view of an exemplary 2D microfluidic device of FIG. 5 in an addressable array of 2×2×1 unit modules.

FIG. 7 is a diagrammatic view of an exemplary 1D microfluidics device including row-wise addressable plumbing with distinct bypass channels and independent side vales, in a 4×4 grid with linear vales 4 and 5 deflated to enable molecular exchange between columns B-N and C-O.

FIG. 8 is a diagrammatic view of a unit element of the plumbing network of an exemplary 1D microfluidics device of FIG. 7.

FIG. 9 is a diagrammatic view of an exemplary 2D microfluidics device including row-column addressable plumbing with distinct bypass channels and independent side valves, in a unit module form.

FIG. 10 is a diagrammatic view of an exemplary 2D microfluidics device of FIG. 9 in an addressable array of 2×2×1 unit modules.

FIG. 11 is a diagrammatic view of an exemplary 2D microfluidics device including row-column addressable plumbing with distinct bypass channels and independent side vales, with emphasis on an addressable chamber/well, flow channels and bypass channels.

FIG. 12 is a diagrammatic view of an exemplary 2D microfluidics device including row-column addressable plumbing with distinct bypass channels and independent side vales, with emphasis on valves blocking flow around each side of a spherical addressable chamber/well.

FIG. 13 is a diagrammatic view of an exemplary 2D microfluidics device including row-column addressable plumbing with distinct bypass channels and independent side vales, with emphasis on valves blocking escape of chemical signals through bypass channels.

FIG. 14 is a diagrammatic view of an exemplary 3D microfluidics device including row-column-stack addressable plumbing with distinct bypass channels and independent side valves, in a unit module form, with three bypasses (all valves omitted for clarity).

FIG. 15 is a diagrammatic view of an exemplary 3D microfluidics device of FIG. 14 including channel-blocking valves, while the bypass-blocking valves are omitted for clarity.

FIG. 16 is a diagrammatic view of an exemplary 3D microfluidics device of FIG. 14 including bypass-blocking valves.

FIG. 17 is a diagrammatic view of an exemplary 3D microfluidics device of FIG. 14 including several unit modules arranged in an arbitrary 2×2×2 array to form a 3D addressable plumbing network.

FIGS. 18A-18D are diagrammatic representations of experiment modes that can be used in some embodiments of the 3D addressable microfluidics device of FIG. 14, with different shades representing different drug and/or cell combinations that can be selectively delivered and/or sampled from each lettered well, including a 2D array (FIG. 18A), a one 3D volume array (FIG. 18B), a 3D array (FIG. 18C), and a 3D sub-volumes array (FIG. 18D).

FIGS. 19A-19H are diagrammatic examples of various valve types capable of being incorporated into an exemplary microfluidic device, including a donut-shaped squeeze valve (FIG. 19A), a square-shaped squeeze valve (FIG. 19B), a classic “Quake” valve (FIG. 19C), a dome-shaped “Quake” valve (FIG. 19D), a ribbon valve (FIG. 19E), a membrane valve (FIG. 19F), a classic squeeze valve (FIG. 19G), and a miniaturized squeeze valve (FIG. 19H).

FIGS. 20A-20C are diagrammatic views illustrating vertical membrane fabrication challenges and a possible solution, including a vertically oriented valve membrane created by fusing horizontal layers of material together (FIG. 20A), the membrane is shown to delaminate at the layer junction due to pressure deforming it from the left side and creating a leak in the plumbing (FIG. 20B), and a reinforcement introduced on the left side of the layers whose inter-layer seams (dashed lines) are purposely offset from those of the membrane (solid line) in order to make delamination less likely (FIG. 20C).

FIG. 21 is a diagrammatic view of an exemplary 3D microfluidics device with miniature squeeze valves.

FIG. 22 is a detailed view of a plumbing modification for maintaining a horizontal squeeze configuration in the exemplary microfluidics device of FIG. 21, which facilitates valve fabrication and includes upper “arms” of the U-shaped part of flow channel in a squeezed configuration.

FIG. 23 is a diagrammatic view of an exemplary 3D microfluidics device with miniature squeeze valves.

FIG. 24 is a detailed view of a plumbing modification for maintaining a horizontal squeeze configuration in the exemplary microfluidic device of FIG. 23, which facilitates valve fabrication and the squeezing action occurs symmetrically on both the upper and the lower “arms” of the U-shaped part of flow channel.

FIGS. 25A and 25B are top and bottom detailed views of a U-shaped squeeze valve from FIGS. 23 and 24 with an added “doormat” element that improves and simplifies blocking of flow.

FIG. 26 is a detailed view of an embodiment of a bypass that includes only straight, right angle channels, which may provide for easier manufacturing due to a lack of diagonal elements or rounded corners.

FIG. 27 is a detailed view of an embodiment of a bypass that includes diagonal orientations relative to the XYZ coordinate axis and rounded corners.

FIG. 28 is a diagrammatic view of a unit module of the 3D addressable plumbing that does not contain any diagonal elements or rounded corners for easier fabrication.

### DETAILED DESCRIPTION

The terminology used herein is to describe particular embodiments only and is not intended to limit the scope of the invention. As used herein, the singular forms “a”, “an” and “the” are intended to include the plural forms as well, unless the context clearly indicates otherwise. Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

In describing the addressability of the devices discussed herein, the following terminology is used to distinguish between row, column and stack addressability. A one-dimensional (1D) device is referred to as “row addressable” and refers to a single pair of flow channels, a two-dimensional (2D) device is referred to as “row-column addressable” and refers to two pairs of flow channels (e.g., along different planes from each other), and a three-dimensional (3D) device is referred to as “row-column-stack addressable” and refers to four pairs of flow channels (e.g., along different planes from each other). This terminology emphasizes that payloads can be addressed and transported across all three dimensions: horizontally (rows), vertically (columns), and through the depth or layers of the array (stacks).

Exemplary embodiments are directed to a high-throughput multiwell addressable microfluidics device. The device can be used within the context of chemical and biological assays but is not limited to such use. The device provides a sophisticated laboratory instrument designed to streamline and enhance the process of conducting a large number of experiments or tests simultaneously (e.g., particularly in the fields of chemistry and biology). The exemplary device allows for efficient movement of payloads in any direction without sacrificing channels or disturbing neighboring payloads.

The device can be structured similarly to a micro-scale version of traditional multiwell plates commonly used in laboratories. However, instead of having a limited number of wells or chambers (like its macro-scale counterpart), the exemplary device includes an array of numerous micro-scale wells or chambers. In some embodiments, such array of micro-scale wells or chambers can be in the hundreds or thousands.

The “high-throughput” aspect of this device, as the term is used herein, refers to the fact that the device can efficiently process a vast quantity of samples, reagents, or conditions concurrently. The device can be customized based on user needs to include any number of wells/chambers, as long as there is external hardware (e.g., pumps, solenoids, pressure regulators, or the like) to operate the number of independent wells. Such high-throughput capability significantly speeds up experimental workflows compared to manual or traditional methods, while simultaneously significantly lowering the costs due to microscopic reagent use and automation. The customization of the device allows for efficient scaling, while lowering the external hardware requirements typically encountered in the industry.

The term “addressable”, as used herein, refers to and highlights a key capability of the device: each individual well or chamber (hereafter used interchangeably and commonly referred to as an “address”) can be independently controlled and manipulated, allowing precise management of fluids, samples, and reactions. This addressable nature of the device allows researchers to tailor specific experimental conditions, such as varying concentrations of substances or different biological samples, to each micro-scale well. This fine-tuned control enables a wide range of experiments to be carried out simultaneously, each with its unique parameters.

The high-throughput multiwell addressable microfluidics devices discussed herein are invaluable tools in various applications (e.g., drug discovery, genomics, proteomics, other areas where the rapid screening and analysis of a large number of conditions or samples are essential, or the like). These devices automate and optimize processes, offering researchers the ability to perform comprehensive assays efficiently, saving time and resources while facilitating groundbreaking discoveries in the chemical and biological sciences.

In some embodiments, the addressable plumbing architecture can be modified or is structured/configured to actuate individual microfluidic ports situated throughout different locations inside of a single large culturing space with the intent of achieving spatiotemporal fluid/cell payload delivery and/or sampling for tissue patterning and nondestructive biological experiment observation. (See, e.g., Tong, A., et al., Automated Addressable Microfluidic Device for Minimally Disruptive Manipulation of Cells and Fluids within Living Cultures. ACS Biomaterials Science & Engineering, 6(3): p. 1809-1820 (2020); and U.S. Pat. No. 11,959,057 to Voronov et al.). In some embodiments, the microfluidics device can be implantable in order to use the addressable ports for providing real-time spatiotemporal information about biomaterial integration inside of a living organism without sacrificing the host. (See, e.g., Nguyen, M., et al., Addressable microfluidics technology for non-sacrificial analysis of biomaterial implants in vivo. Biomicrofluidics, 17(2) (2023)). In some embodiments, the addressable microfluidics devices can serve as support structures (e.g., scaffolds) for tissue growth, in which case the devices can be biodegradable. (See, e.g., Tong, A. et al., A Minireview of Microfluidic Scaffold Materials in Engineering Engineering, Front. Mol. Biosci., Sec. Biophysics (2022)). Therefore, the addressable microfluidics devices discussed herein have a wide range of applications.

As noted herein, the traditional microfluidics devices have been labeled as either having a “Row-Wise Addressable Plumbing Layout with Distinct Bypasses” design, or a “Row-Column Addressable Plumbing Layout with Sacrificial Bypass Columns” design. The exemplary device discussed herein can be labeled as a “Row-Column-Stack Addressable Plumbing Layout with Distinct Bypasses and Independent Side Valves”.

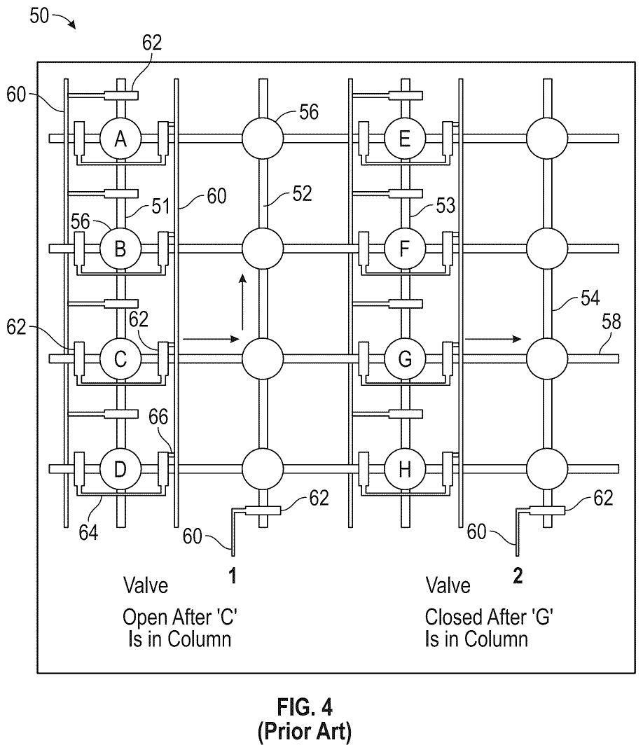

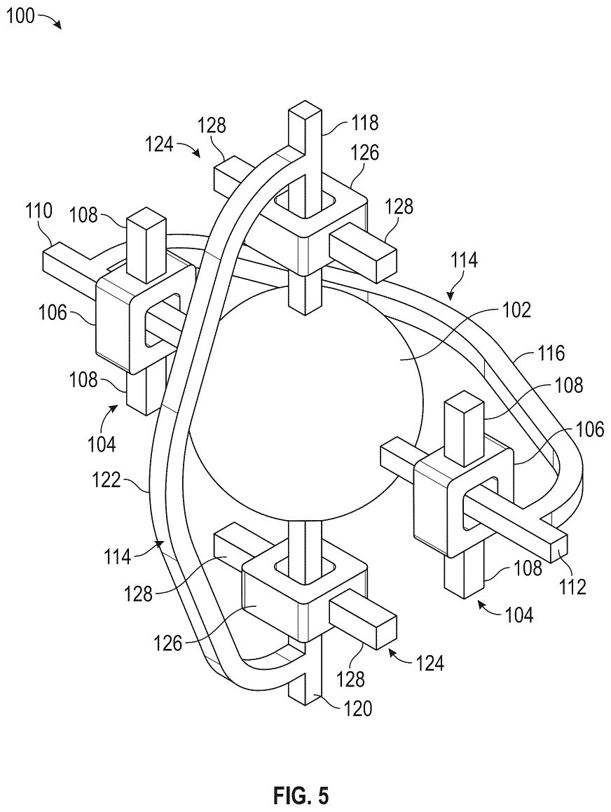

The traditional microfluidics devices discussed herein are generally limited by a two-dimensional (2D) structure due to the fabrication methods commonly available in the industry. These fabrication methods typically involve bonding together separate layers produced via lithography and/or etching. In contact, the exemplary microfluidics devices have a three-dimensional (3D) structure that takes advantage of the plumbing layouts of FIGS. 3 and 4, but without their drawbacks. In some embodiments, the exemplary devices can be fabricated using 3D printing, thereby generating the entire device structure without the need for stacking 2D layers. However, the present disclosure is not limited to formation by 3D printing and, instead, other fabrication methods can be used to achieve the 3D structure of the exemplary devices. For example, the exemplary devices could be manufactured using traditional methods and assembled in a 3D configuration to achieve the 3D structure and advantageous operational improvements discussed herein.

Critical to fabrication of the device is the need to have flexible membranes for the on-chip valves, while the structural portions of the device may be form-holding. In some embodiments, the form-holding layers can be fabricated separately (e.g., via 3D printing, laser cutting/etching, lithography, molding, sacrificial templating/gel casting, combinations thereof, or the like), and thin film layers can subsequently be sandwiched between them to serve as the membrane. The layers can be stacked, aligned and bound together using various methods (e.g., glue, heat, bolts, press, plasma surface treatment, combinations thereof, or the like). However, in other embodiments, it is possible to fabricate both the form-holding and flexible parts of the device in one process using fabrication technologies such as, but not limited to, 3D printing. This can be accomplished by varying printer settings and/or materials in order to achieve flexible properties in one area and form-holding properties in another area as the device is being formed. Alternatively, a sacrificial template of the device's inverse geometry could be 3D printed and used for gel casting the device (with the template eventually washed out from its negative spaces). Such 3D printing process provides optimal precision and automation for fabrication of the exemplary devices.

The exemplary devices allow for fluid, chemical or cell exchange between addressable wells in the horizontal (rows), vertical (columns), and depth or layers of the array (stacks). The devices are therefore capable of payload exchange between individual wells/locations without the payload passing through intermediate-to-designation wells. As such, the devices prevent mixing of contents within intermediate wells (as is possible in traditional devices), and do not sacrifice wells in the array. The ability to pass payloads horizontally, vertically, and through the depth or layers of the array (e.g., three-dimensional payload transfer) provides improved efficiency for use of the device. The devices allow for such transfer while also maintaining the bypass ability that allows payloads to not mix with contents of the intermediate wells, and without the sacrifice of wells.

The addressable microfluidic device can be used for precise control and manipulation of small volumes of fluids, often at the microliter or nanoliter scale. The device allows for the automated and programmable delivery of different payloads (such as fluids, cells, chemicals) to specific locations within the device. The device includes a chip that has a network of microscopic channels, chambers/wells, and valves integrated into it. The chip can be made using different technologies, including (but not limited to) 3D printing. The chambers are distributed in a grid within the chip and can be used to store cells, fluids and/or chemicals, perform culturing experiments, drug screenings, and chemical reactions using microscopic amounts of reagents.

Each chamber is individually addressable, meaning that payloads can be delivered to any individual chamber in the device. This is accomplished by selectively blocking off undesired chambers from the flow channels using valves and rerouting the payloads via bypass channels that travel around the chambers. On the other hand, the payloads are delivered to the desired chambers by disabling a valve around them and thereby opening their access to the flow channels carrying the payloads. Since the payloads are rerouted via the bypass channels around the undesired chambers, the payloads do not need to pass through intermediate wells on the way to their destinations. The system includes external electronics, pneumatics and software synching all components together to drive the flow action on the chip.

The device is therefore a high throughput microfluidics device capable of transferring sample payloads horizontally (rows), vertically (columns), and through the depth or layers of the array (stacks) while bypassing non-targeted wells. The payloads can therefore be transferred between wells across rows, columns and stacks of the device without passing through intermediate wells to reach their destination and without mixing. The device allows for high throughput combinatorial chemical reactions and/or cell culture screening. The device allows for co-culturing different cell types with interactions between adjacent wells. The device can have a high density and allows for versatile experimental flexibility. The device includes plumbing architecture that incorporates bypass channels in each of the horizontal, vertical, and depth/layer directions, as compared to traditional devices that either have no bypass channels, the bypass channels are situated only in the horizontal direction, or wells act as sacrifice channels to serve as bypasses (which wastes device space and reduces the device experimental throughput).

### Microfluidics Device (100)

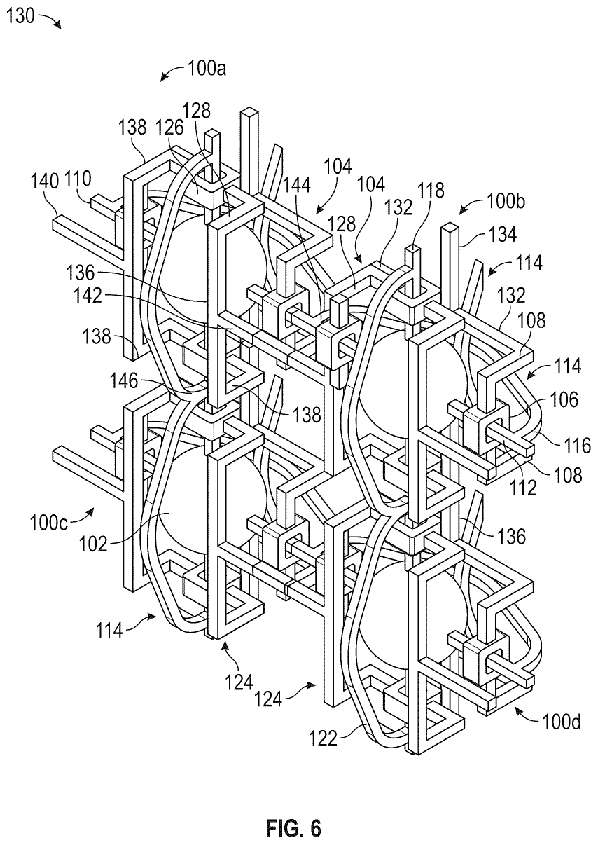

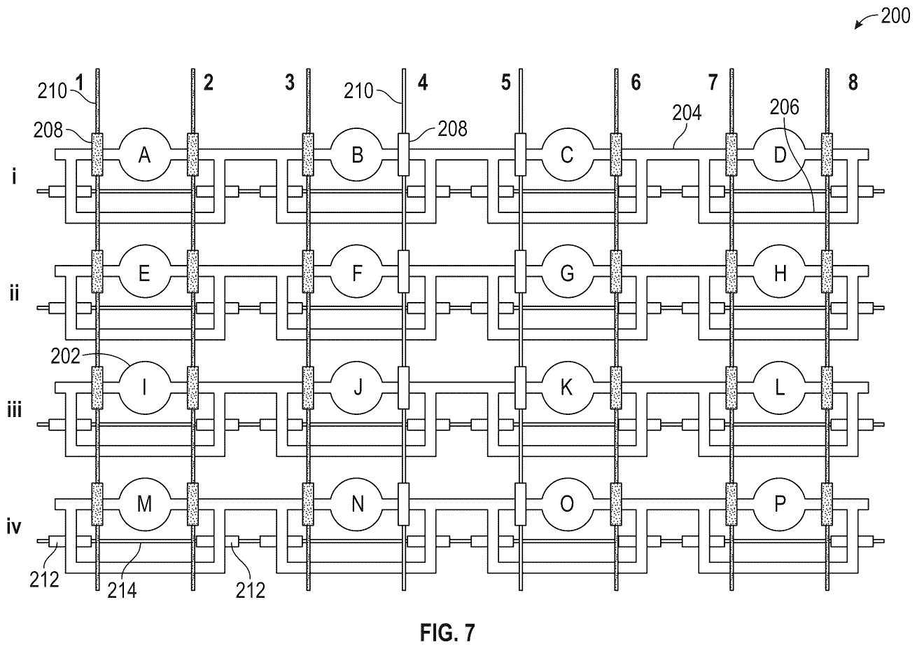

FIG. 5 shows a diagrammatic view of an exemplary microfluidics device 100 having a three-dimensional structure which provides addressability with an efficient design, including flow in a 2D configuration. The device 100 can represent a single unit module of a “Row-Column Addressable Plumbing Layout With Distinct Bypasses” system. FIG. 6 shows multiple devices 100a, 100b, 100c, 100d coupled together in fluid communication with each other, thereby forming a 2×2×1 array or system of unit modules. However, it should be understood that any number of the unit modules could be coupled together to define a system of modules for improved assay operation.

The device 100 includes a well 102 (e.g., a spherical cell culture chamber for chemical reactions, an address, or the like). Although shown in a spherical configuration, it should be understood that the well 102 can be of any configuration. In the context of microfluidics, the preference of cells for spherical versus cylindrical wells can depend on various factors, including cell type, the specific application, and the microenvironment requirements. Generally, spherical wells can provide a more homogenous environment with uniform cell distribution and nutrient diffusion, potentially promoting more natural cell behaviors and interactions. This can be particularly advantageous for 3D cell cultures and tissue engineering applications, where mimicking the in vivo environment is crucial. Cylindrical wells (which is more traditional microfluidics), on the other hand, are often used for ease of fabrication and handling. Such cylindrical wells can be more straightforward to produce and integrate into microfluidic devices. It should be noted that any of the exemplary devices discussed herein can therefore include various well configurations.

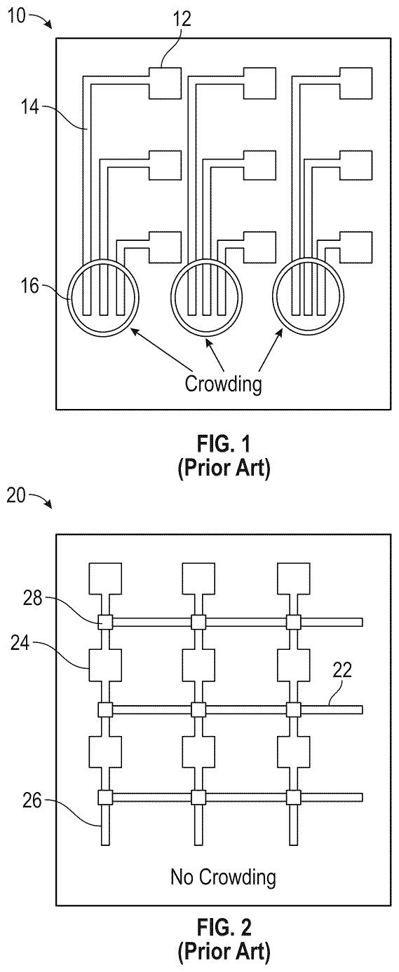

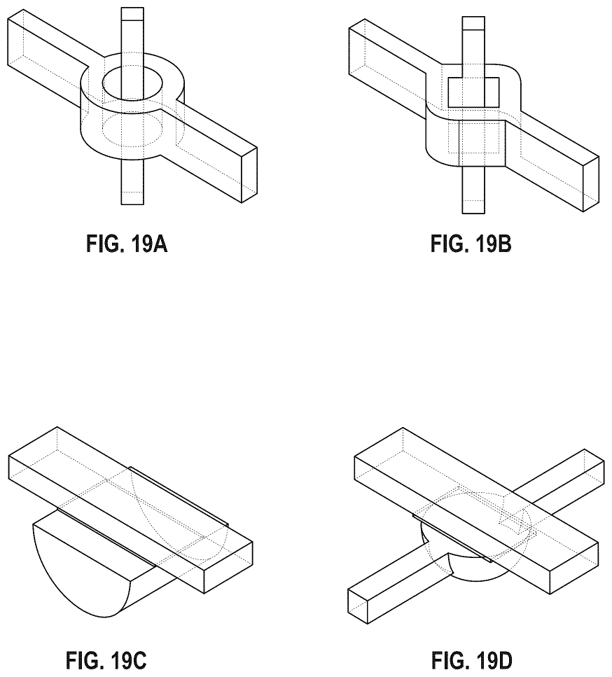

The device 100 includes two row valve assemblies 104 (e.g., including the side valve 106 and associating piping or blocking channels 108) for blocking or opening flow to/from the well 102 via the horizontal channels 110, 112 (e.g., row flow channels). It should be understood that different types of valve assemblies can be used with the exemplary devices discussed herein (i.e., not limited to device 100), with such valve assemblies being interchangeable (as discussed with respect to FIG. 19), and the figures provide only non-limiting examples of such valve assemblies in the form of “squeeze” valves. (See, e.g., FIG. 1 of Noriega, J. L. S. et al., Spatially and optically tailored 3D printing for highly miniaturized and integrated microfluidics, Nature Communications, 12:5509 (2021)). The valves 106 are positioned around the respective flow channels 110, 112. The device 100 includes bypass assemblies 114 for routing flow around the well 102 when the valves 106 are closed. The bypass assemblies 114 includes a horizontal assembly including the horizontal channels 110, 112, which are fed by and coupled to connecting channel 116; and a vertical assembly including vertical or orthogonal channels 118, 120, which are fed by and coupled to connecting channel 122. The connecting channel 116 therefore connects the channels 110, 112 to each other; and the connecting channel 122 similarly connects the channels 118, 120. The device 100 includes column valve assemblies 124 for blocking or opening flow to/from the well 102 via the orthogonal channels 118, 120 (e.g., column channels). Each valve assembly 124 includes a side valve 126 and associated piping or blocking channels 128. The valves 126 are positioned around the respective channels 118, 120. The side valves 106 for flow channels 110, 112 can be actuated into open or closed positions in a simultaneous manner (e.g., if the valve 106 for channel 110 is closed, the valve 106 for channel 112 is also closed). Similar operation is used for valves 126 for flow channels 118, 120. The valves 106, 126 therefore operate together in a joined manner with their respective pair.

The valve assemblies 104 are therefore positioned on opposite sides of the well 102 relative to each other (e.g., 180° and along the same plane), the valve assemblies 124 are positioned on opposite sides of the well 102 relative to each other (e.g., 180° and along the same plane), the planes of the valve assemblies 104, 124 are the same, and the valve assemblies 124 are radially offset from the valve assemblies 104 by about 90°. The channels 110, 112 of the bypass assembly 114 extend along the same plane on opposite sides of the well 102, and the channels 118, 120 similarly extend along their respective same plane on opposite sides of the well 102, with the pairs of channels 110, 112 and channels 118, 120 orientated about 90° from each other. The connecting channels 116, 122 extend in opposite directions from the well 102 and are oriented along perpendicular intersecting planes. However, it should be understood that the 90° arrangement can be varied and flow channels and/or valve assemblies to do need to be oriented in such positioned relative to each other. The full plumbing that actuates the valves 106, 126 is only shown partially for clarity in FIG. 5, but is shown fully in the system view of FIG. 6. The piping depicted in FIGS. 5 and 6 is smaller than the actual valves 106, 126, so as to minimize the interference with other elements of the device 100 (as it has to be inflated and deflated to close and open the valve, respectively).

FIG. 5 therefore shows the three-dimensional representation of a “Row-Column Addressable Plumbing Layout with Distinct Bypasses” device 100. Specifically, FIG. 5 shows a close-up or detailed view showing a unit module of the device 100 that can be repeated throughout an addressable array, with valve plumbing omitted for clarity. FIG. 6 shows an addressable array 130 including 2×2×1 unit modules (i.e., devices 100). In FIG. 6, a part of the bypass assembly 114 is shown to originate from behind the array 130, belonging to a neighboring address (e.g., well 102) that is not shown (for example, if the system was 2×2×2 in size, as opposed to the 2×2×1 depicted in FIG. 6) to demonstrate how the bypass assemblies 114 overlap each other in 3D space to make the overall system more compact. For the 2×2×1 size addressable array 130 provided in FIG. 6, the first dimension is chosen arbitrarily and the second dimension is chosen to have unity with the first dimension for clarity, but in principle can also be of any size. All addresses are capable of containing payloads simultaneously while maintaining the full fluid routing capability.

With focus on FIG. 6, the adjacently positioned devices 100 (whether positioned adjacently over each other (vertically) or next to each other laterally (horizontally)) are connected to each other with plumbing or channels that allow for fluid interconnection of similar components. Valve assemblies 104 within each device 100 are therefore connected to each other via plumbing, and are similarly connected on opposing sides to valve assemblies 104 of the adjacent devices 100. Valves assemblies 124 are similarly connected within each device 100, and are connected on opposite sides to valve assemblies 124 of the adjacent devices 100. Bypass assemblies 114 are also connected within each device 100, and are connected on opposite sides to bypass assemblies 114 of the adjacent devices 100.

For example, the blocking channels 108 associated with the valve assemblies 104 connect to additional channels 132, which join the two valve assemblies 104 of each device 100. The channels 132 also lead to an inter-device connection channel 134, 136 extending either vertically upward away from the well 102 or vertically downward away from the well 102 to connect to valve assemblies 104 of the adjacently positioned devices 100. In a similar manner, the blocking channels 128 associated with the valve assemblies 124 connect to additional channels 138, which join the two valve assemblies 124 of each device 100. The channels 138 also lead to an inter-device connection channel 140, 142 extending either horizontally leftward away from the well 102 or horizontally rightward away from the well 102 to connect to valve assemblies 124 of the adjacently positioned devices 100.

The bypass assemblies 114 are similarly interconnected between adjacently positioned devices 100 of the array 130. As discussed above, the connecting channel 116 connects to the channels 110, 112 on opposing sides of the well 102. In the array 130, the connecting channels 116 of adjacent devices 100 extend along the same plane and connect to each other at a joint 144 which, in turn, creates a connection between the channel 112 of one device 100a with the channel 112 of the adjacent device 100b. As discussed above, the connecting channel 122 connects to the channels 118, 120 on opposing sides of the well 102 (see FIGS. 5 and 6). In the array 130, the connecting channels 122 of adjacent devices 100 extend along the same plane and connect to each other at a joint 146 which, in turn, creates a connection between the channel 120 of one device 100a with the channel 118 of the adjacent device 100c (see FIGS. 5 and 6).

Although the plumbing/channels connecting the devices 100a, 100b, 100c, 100d to each other are shown as making substantially 90° turns, it should be understood that a more curved configuration of the interconnecting plumbing/channels can be used. However, the interconnection of the same components between adjacently positioned devices 100a, 100b, 100c, 100d remains the same. The example shown in FIG. 6 is merely used to illustrate the ability to interconnect multiple devices 100 to each other, showing the ability of the array 130 to be scalable while providing for addressability.

In the “Row-Column Addressable Plumbing Layout with Distinct Bypasses” configuration, fluids carrying cellular and/or chemical payloads can travel both horizontally and orthogonally either through the bypass assemblies 114 or via the flow channels 110, 112, 118, 120 to the culture chamber/wells 102 (see FIGS. 5 and 6). For example, opening of valve 106 at channel 110 allows flow into or out of the well 102 via channel 110. As another example, opening of the valve 106 at channel 112 allows flow into or out of the well 102 via channel 112. Closure of the valves 106 at channels 110, 112 bypasses flow via bypass channel 116. Similarly, opening of the valve 126 at channel 118 allows flow into or out of the well 102 via channel 118. As another example, opening of the valve 126 at channel 120 allows flow into or out of the well 102 via channel 120 (see FIGS. 5 and 6). Closure of the valves 126 at channels 118, 120 bypasses flow via bypass channel 122. Therefore, the payloads can be either transferred to, or can bypass, any adjacent neighbor without the need for sacrificial channels or having to move chamber contents out of the way (as was the case for the “Row-Column Addressable Plumbing Layout with Sacrificial Bypass Columns” traditional configuration in FIG. 4). The device 100 configuration therefore results in a more functional and denser populated high throughout experimentation microfluidics device with efficient structure and operability.

One main advantage of the device 100 is being able to mix well contents in both the horizontal and the orthogonal directions. For example, all of the wells 102 can be loaded with different “payloads” (depending on what the application is, these could be cells, bacteria, yeast, virus, DNA, drugs, or just chemicals), and then some time is allowed for a reaction or process take place. After that, the well contents are mixed in either horizontal or in orthogonal, or in both directions. This can be done over and over as many times as needed.

In some embodiments, the device 100 could optionally be used as a traditional addressable device, where the contents of each well stay in that well for the duration of the experiment (essentially a microscopic analogue for a multi-well plate). However, it would still be faster to load and unload the device 100 (as compared to traditional devices), since contents could be sent to/from wells in both the horizontal and the orthogonal directions (instead of just horizontal and then snake around the device to get to where you need to be). The device 100 is therefore a high throughput experimentation device with an additional flexibility and speed relative to the traditional device.

### Microfluidics Device (200)

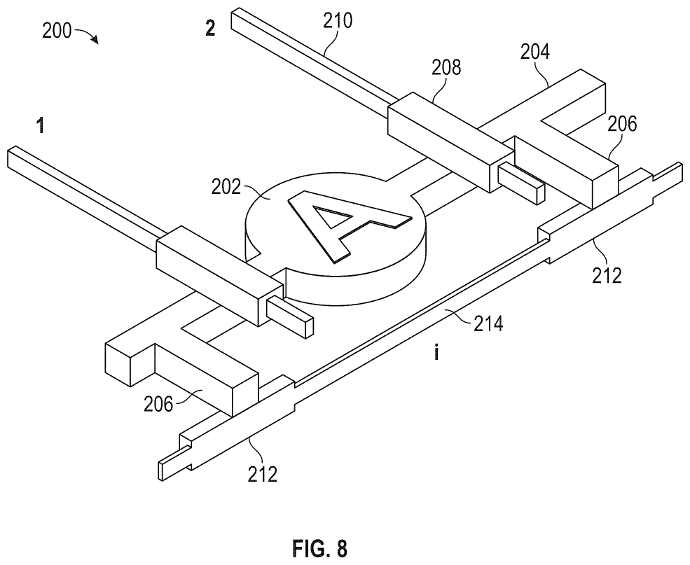

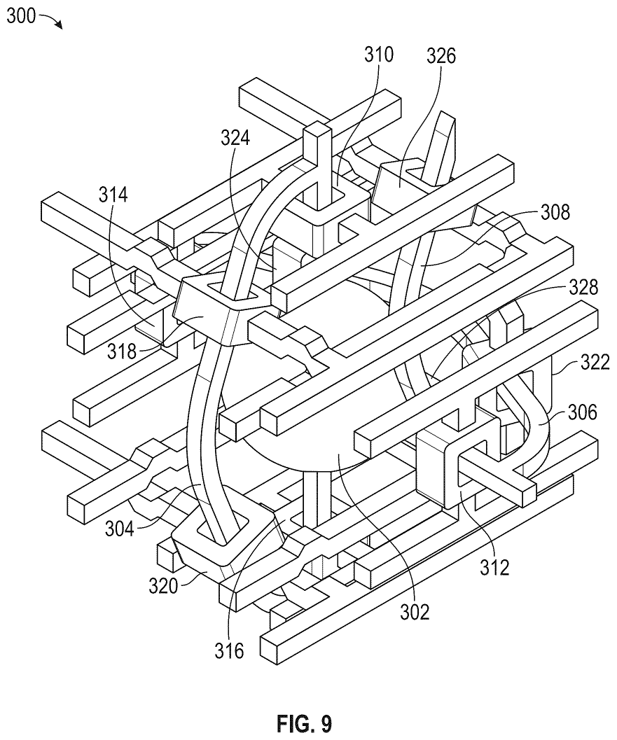

FIGS. 7 and 8 provide a diagrammatic view of an exemplary addressable microfluidics device 200 which includes a 1D flow configuration. The device 200 allows for inter-address molecular communication in the absence of flow. In particular, the device 200 includes row-wise addressable plumbing layouts with distinct bypass channels and independent side valves. FIG. 7 shows a 4×4 array/grid representative of the device 200.

The device 200 includes wells 202 labeled with capital letters (e.g., A, B, C, D, etc.). The device 200 includes rows of flow channels 204 passing through groups of wells 202. For example, the first row of flow channels 204 is fluidly connected to wells 202 labeled A, B, C, D; and the second row of flow channels 204 is fluidly connected to wells 202 labeled E, F, G, H. Around each well 202 and fluidly connected to the flow channels 204, the device 200 includes bypass channels 206.

The device 200 includes orthogonal valves 208 on either side of the well 202 and configured to be actuated to open to allow flow through the channel 204, or close to prevent flow through the channel 204 to the well 202 (thereby forcing flow through the bypass channel 206 around the well 202). The blocking channels 210 associated with the column of valves 208 are labeled with Arabic numerals. The valves 208 are either inflated to close the channel 204 or deflated to open the channel 204. As used in FIG. 7, a solid or filled in rectangle is representative of an inflated or closed valve 208, while a hollow rectangle is representative of a deflated or open valve 208. The respective columns of valves 208 are independently actuated, although opening or closing occurs for the entire column (e.g., if column 1 is closed, all valves 208 in column 1 are closed). The device 200 includes linear valves 212 extending perpendicularly relative to the orthogonal valves 208. The linear valves 212 are connected to each other by blocking channels 214, with the group of valves 212 labeled using Roman numerals. The bypass channels 206 each include two linear valves 212 on either side of the channel 206 to allow or prevent flow through the respective bypass channels 206. The valves 212 can be inflated or deflated to close or open flow through the bypass channel 206.

As shown in FIG. 7, the linear valves 212 of rows 4 and 5 have been deflated to open the valves 212, thereby enabling molecular exchange between columns of wells 202 labeled B-N and C-O. As noted herein, the linear valves 212 block the spread of the molecular signals through the bypass channels 206, while the orthogonal valves 208 establish a communication between the neighboring addresses or wells 202 in the absence of flow.

In some situations, it may be desirable to enable molecular communication between neighboring addresses in the absence of flow, for example between the wells or locations B and C in the modified version of the “Row-wise Addressable Plumbing Layout with Distinct Bypasses” shown in FIG. 7 (termed here as “Row-wise Addressable Plumbing with Distinct Bypasses and Independent Side Valves”). This can be useful for establishing interactions between heterogeneous cell cultures or for simply allowing chemical reactants stored in the neighboring wells to diffuse between them. In that case, each of the addressable wells can be equipped with orthogonal linear valves 212 labeled as Arabic numerals 1-8 that can either open or close each side of the chamber independently of the other, although opening or closing occurs for the entire column of valves 208. The linear valves 212 are therefore operable independently of each other, with two linear valves 212 for each bypass channel 206.

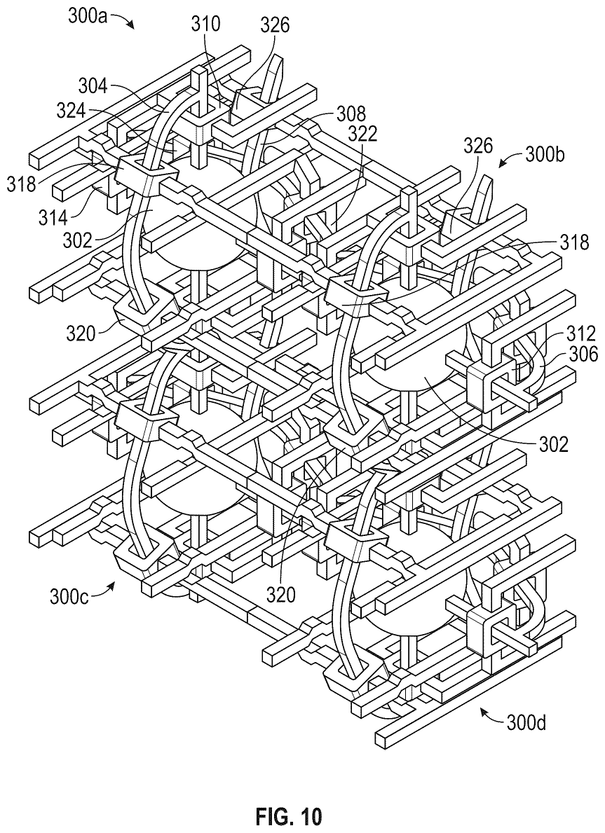

Such independent operation can be visualized with an example. If there are two different cell cultures in neighboring wells: healthy cells in well ‘B’ and cancer cells in well ‘C’. A study is desired to determine how the molecular signals from the cancer cells affect the healthy cells. To do this, valves 4 and 5 are opened, such that the molecular signals can be exchanged between the two cell types via diffusion. However, it is not desired for the molecular signals to travel into the bypass and contaminate the rest of the system. For this reason, the horizontal valve ‘I’ is used, which will block all bypasses 206 in the first row of addresses. In addition, opening column valves 4 and 5 establishes communication not only between wells ‘B’ and ‘C’, but between the entire columns of wells ‘B’-‘N’ & ‘C’-‘O’. This may be desired if there are replicate experiments in rows 2-4; or it may not be desired if enablement of an exchange between wells ‘B’ and ‘C’ only is desired, and no other wells (for the latter case, the device 300 of FIG. 9 can be used). Furthermore, another set of horizontal linear valves 208 labeled as Roman numerals i-iv is added below the main plumbing (see detailed view of FIG. 8) to block the spread of the chemical signals through the bypass channels 206 while the neighboring addresses or wells 202 are open to each other.

In order to achieve molecular communication between the addresses B and C only, the linear valves 212 labeled as rows 4 and 5 in-between these wells 202 can be set to an open state as shown in FIG. 7, while the linear valves 212 in rows 3 and 6 on their outer edges can be set to a closed state. Additionally, the horizontal valves 212 in row i can also be actuated to close in order to block the spread of the chemical signals B and C through the bypass channels 206. Furthermore, given that the linear valves 212 actuate entire columns of addresses (wells 202), the act of opening the linear valves 212 of rows 4 and 5 would also similarly establish communication between addresses F and G, J and K, and N and O in FIG. 7. Therefore, the bypass-blocking horizontal valves 212 of rows ii-iv would also be inflated to close in the example shown in FIG. 7.

The device 200 therefore provides several advantages over traditional devices. In particular, the device 200 establishes a connection between neighboring columns of addresses in order to enable diffusive (i.e., not flow-driven) exchange of a chemical. One application for the device 200 is to study cell co-cultures (as described previously). In cases where cells are migratory and it is not desired for them to mix, a modified setup can be used, where three columns are in communication with each other: the two side columns hold the different cell types (as in the previous example) while the middle column holds some kind of a barrier (e.g., gel or mesh) that prevents migration (but still allows diffusion). It should be understood that the device 200 can be used for other non-bio applications, such as putting two different chemicals into the neighboring wells and studying diffusion-driven reactions. The vertical valves of the device 200 can be actuated ‘on’ and ‘off’ over time to enable dynamic experiment variations.

### Microfluidics Device (300)

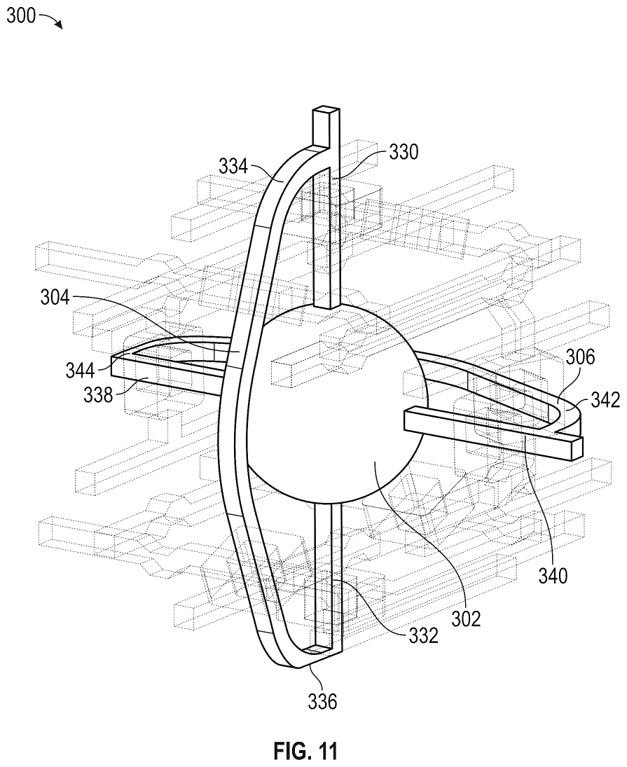

It may not always be desirable to establish molecular communication between the entire columns of addresses (as was described in the example above for FIG. 7). To that end, the “Row-Column” equivalent of this design is shown in FIGS. 9 and 10. In particular, FIG. 9 shows a unit module (i.e., the microfluidics device 300) which includes a chamber or well 302 (e.g., a spherical well), flow channels 304, 306, 308 (with channel 308 being a neighboring unit module channel); flow channel-blocking valves 310, 312, 314, 316 (e.g., side valves); and bypass-blocking valves 318, 320, 322, 324, 326, 328 (with valves 326, 328 belonging to a neighboring unit module) (e.g., bypass valves). The microfluidics device 300 defines a 2D flow configuration. The device 300 can be substantially similar to the device 100, except for the distinctions noted here. For example, rather than including only side valves, the device 300 also includes bypass valves, which allows the side valves to operate into open/closed positions independently from their respective pair.

FIG. 10 shows how four of these unit modules or devices 300a, 300b, 300c, 300d can be fluidly and operationally connected together in a 2×2×1 array of addresses (although any number of devices 300 could be assembled in a similar manner). Similar to the previous configurations of the exemplary device discussed herein, when devices 300a, 300b, 300c, 300d are positioned adjacent to each other, similar components are linked together fluidly. As used herein, the term “adjacent” refers to neighboring devices that are connected to each other along the same plane of the page in both horizontal and vertical directions, as well as devices connected to each other into and out of the page direction. Thus, bypass connecting channels 304 (and similarly flow channel 306) are fluidly joined to flow channels 304 of the adjacent device 300. Further, channels associated with the flow channel-blocking valves 310 (and similarly valves 312, 314, 316; see, e.g., FIGS. 9 and 10) are fluidly joined to channels for valves 310 of the adjacent device 300. Further, channels associated with the bypass-blocking valves 318, 326 (and similarly valves 320, 322, 324, 328; see, e.g., FIGS. 9 and 10) are fluidly joined to channels for valves 318, 326 of the adjacent device 300.

With reference to FIGS. 9 and 10, the flow channel 304 provides for flow into the well 302 on opposite sides of the well 302 (e.g., top and bottom sides). The flow channel-blocking valves 310, 316 (e.g., side valves) are configured to open and close to regulate flow to the well 302, or force flow through the bypass channel associated with the flow channel 304. For example, the valve 310 blocks the upward flow channel. The valves 310, 316 are operable independently of each other, allowing for selective opening and closing. The bypass blocking valves 318, 320 (e.g., bypass valves) are associated with the bypass channel of the flow channel 304, and are configured to open and close to regulate flow through the bypass channel.

The flow channel 306 provides for flow into the well 302 on opposite sides of the well 302 (e.g., right and left sides), with the connections to the well 302 radially oriented about 90° relative to the inlets of the flow channel 304. The flow channel-blocking valves 312, 314 are configured to open and close to regulate flow to the well 302, or force flow through the bypass channel associated with the flow channel 306. The valves 312, 314 are operable independently of each other, allowing for selective opening and closing. The bypass blocking valves 322, 324 are associated with the bypass channel of the flow channel 306, and are configured to open and close to regulate flow through the bypass channel. The flow channel 308 and the bypass-blocking valves 326, 328 are associated with the next adjacent device 300, and are equivalent to the flow channel 304 and bypass-blocking valves 318, 320.

FIGS. 9 and 10 therefore show a “Row-Column Addressable Plumbing With Distinct Bypasses and Independent Side Valves” configuration for the device 300: As noted herein, a part of a bypass channel 308 is shown to originate from behind the array, belonging to a neighboring address that is not shown (e.g., if the system was 2×2×2 in size, as opposed to the 2×2×1 array of FIG. 10) to demonstrate how the bypasses overlap each other in 3D space to make the device 300 more compact. The sphere represents the well 302 in which chemical reactions and/or cell cultures occur. All addresses (wells 302) are capable of containing payloads simultaneously while maintaining the full fluid-routing capability.

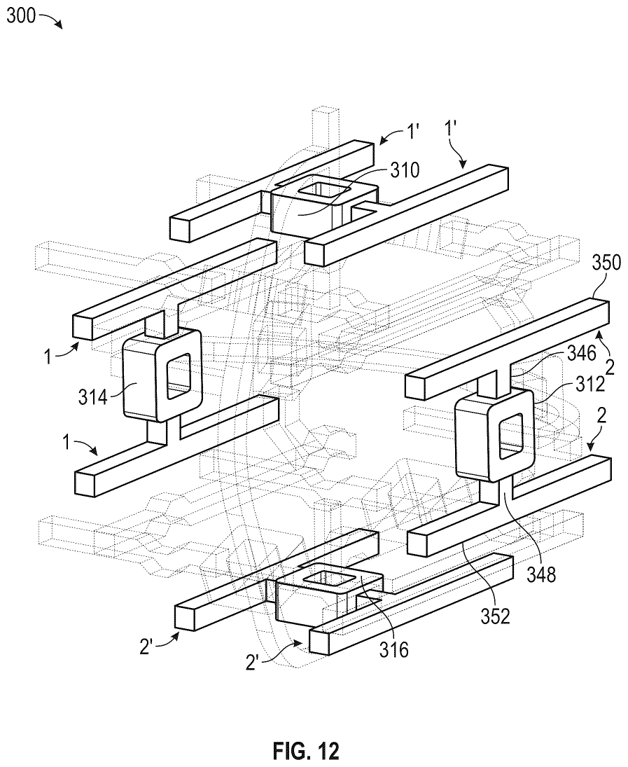

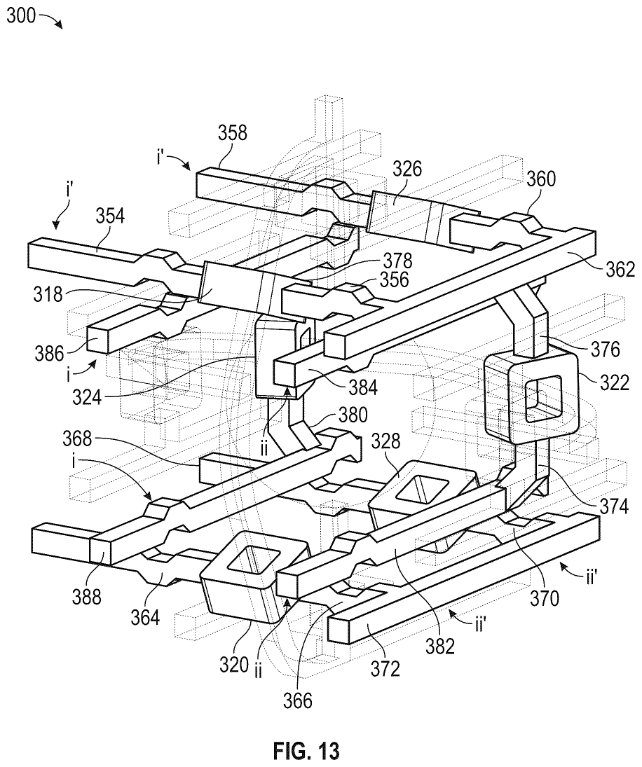

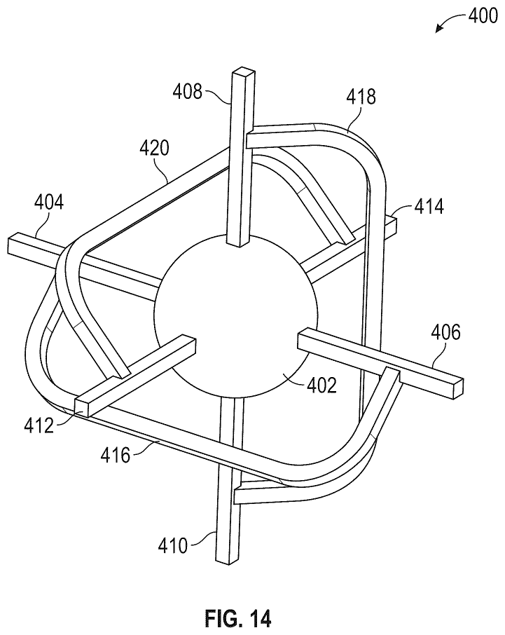

FIGS. 11-13 show detailed sections of the device 300 for clarity. In particular, FIG. 11 shows the well 302 with the flow channels 304, 306; FIG. 12 shows the flow channel blocking valves 310, 312, 314, 316 and their associated channels; and FIG. 13 shows the bypass channel blocking valves 318, 320, 322, 324, 326, 328 and their associated channels. FIG. 11 therefore shows a spherical chamber/well 302 with flow channel blocking and bypass blocking plumbing in transparent lines for context. FIG. 12 shows linear valves that block the flow channels on each side of the well 302 independently of the other. Given that flow could occur in either the horizontal or the orthogonal direction, the valves labeled without primes denote the former, while the ones with the primes denote the latter (1, 1′, 2, 2′, etc.). The remainder of the device in FIG. 12 is drawn in transparent lines for context. FIG. 13 shows the valves used for blocking the escape of chemical signals through the bypass channels, when a respective valve from FIG. 12 is opened to enable communication with a neighboring address. The same prime convention is used in FIG. 13 as in FIG. 12 (i, i′, ii, ii′, etc.) Specifically, in these FIGS. 12 and 13, the valves labeled using the Arabic numerals 1, 2, 3 and 4 are used to close/open the flow channels, while the valves labeled using the Roman numerals i, ii, iii and iv are used to close/open the bypass-blocking valves, respectively. Numbers without primes denote blocking of flow in the horizontal direction, while those with the primes denote blocking of flow in the orthogonal direction. Plumbing elements not emphasized are made partially transparent for clarity and for context.

With reference to FIG. 11, the flow channel 304 includes input channels 330, 332 on opposite sides of the well 302 (e.g., top and bottom sides). The input channels 330, 332 are fluidly connected to each other by a bypass channel 334. A joint 336 provides a fluid connection with the flow channel 304 of the next adjacent device 300 of the array. Similarly, the flow channel 306 includes input channels 338, 340 on opposite sides of the well 302 (e.g., right and left sides), orientated 90° radially relative to the input channels 330, 332. The input channels 338, 340 are fluidly connected to each other by a bypass channel 342. A joint 344 provides a fluid connection with the flow channel 306 of the next adjacent device 300 of the array. The bypass channels 334, 342 are oriented transverse and perpendicular relative to each other.

With reference to FIGS. 11 and 12, each of the flow channel blocking valves 310, 312, 314, 316 is shown. The valves 310, 316 regulate flow of the input channels 330, 332 (shown in FIG. 11), respectively, while the valves 314, 312 regulate flow of the input channels 338, 340 (shown in FIG. 11), respectively. Each valve 310, 312, 314, 316 includes intermediate channels 346, 348 extending from opposite sides, and connecting channels 350, 352 extending in opposite directions from the intermediate channel 346, 348 ends. The connecting channels 350, 352 allow fluid connection to connecting channels 350, 352 of the adjacent devices 300.

With reference to FIG. 13, each of the bypass blocking valves 318, 320, 322, 324, 326, 328 is shown. The valves 318, 320 regulate flow through the bypass channel 334 (shown in FIG. 11); the valves 326, 328 regulate flow through the bypass channel 334 of the adjacent device 300; and the valves 322, 324 regulate flow through the bypass channel 342 (shown in FIG. 11). The valve 318 includes intermediate channels 354, 356 extending from opposite sides, and the valve 326 similarly includes intermediate channels 358, 360 extending from opposite sides. The intermediate channels 354, 358 connect to similar channels of the adjacent device 300, while the intermediate channels 356, 360 connect to a connecting channel 362 that fluidly connects the valves 318, 326. The connecting channel 362 may be optional in case the actuation of the bypass blocking valve 318 is to be done independently of the analogous valve 326 belonging to the adjacent device 300. The valves 320, 328 similarly include intermediate channels 364, 366, 368, 370 extending from opposite sides, with channels 366, 370 connected to a connecting channel 372 that fluidly connects the valves 320, 328. The connecting channel 372 may be optional in case the actuation of the bypass blocking valve 320 is to be done independently of the analogous valve 328 belonging to the adjacent device 300. Similarly, the valves 322, 324 includes intermediate channels 374, 376, 378, 380 extending from opposite sides, with each channel 374, 376, 378, 380 connected to a respective intermediate channel 382, 384, 386, 388 that fluidly connects the valves 322, 324 to other similar valves 322, 324 of adjacent devices. The device 300 is therefore addressable in both the horizontal and the orthogonal directions (while the device 200 was only addressable in the horizontal direction).

Overall, FIGS. 7-8 and FIGS. 9-13 show “Row-wise. . . ” and “Row-Column Addressable Plumbing with Distinct Bypasses and Independent Side Valves”, respectively. These microfluidics device 200, 300 configurations can be used to establish communication between neighboring chambers/wells in the absence of flow. Furthermore, in some embodiments, instead of establishing communication between just two adjacent neighbors, there could instead be three or more addresses in direct communication with each other. An example of this application can be illustrated by imagining that addresses A and C in FIG. 7 are loaded with two different cell types exchanging chemical signals with each other, while the address B in-between them is loaded with some type of a mesh or a gel for preventing inter-well migration. This way the two cultures can be kept separate, but still interacting with each other. Thus, the independent side valves offer greater flexibility to the types of experiments that can be run in the high-throughput microfluidics devices.

The devices discussed herein can be used for a variety of applications. For example, the applications that utilize channels opening between neighboring addressable wells to establish molecular communication via diffusion in the absence of flow can include (but are not limited to), e.g., synthetic biology and genetic circuit testing, neural network mimicry, gradient formation for chemotaxis studies, metabolic interaction studies, synthetic tissue engineering, drug testing and interaction studies, quorum sensing in bacterial cultures, immune cell interactions, developmental biology, sensor networks for environmental monitoring, or the like. These applications demonstrate the potential of addressable microfluidic systems with diffusion-based communication to advance research in various fields by enabling precise control and study of molecular interactions and signaling pathways.

Synthetic Biology and Genetic Circuit Testing: The application includes testing interactions between synthetic genetic circuits. The unique feature includes addressable wells that allow controlled diffusion of signaling molecules between different genetic constructs, enabling precise study of intercellular communication.

Neural Network Mimicry: The application includes simulating neural communication and synaptic signaling. The unique feature includes opening channels between wells containing neurons or neural cells that permits diffusion of neurotransmitters, allowing the study of neural network behaviors and synaptic plasticity.

Gradient Formation for Chemotaxis Studies: The application includes investigating cell movement in response to chemical gradients. The unique feature includes diffusive communication between wells that enables the creation of stable chemical gradients, facilitating the study of chemotactic responses in bacteria, immune cells, and cancer cells.

Metabolic Interaction Studies: The application includes exploring metabolic exchanges between different cell types. The unique feature includes addressable wells that allow the diffusion of metabolites and signaling molecules between co-cultured cells, providing insights into symbiotic relationships and metabolic dependencies.

Synthetic Tissue Engineering: The application includes developing complex tissue structures with intercellular communication. The unique feature includes diffusion channels between wells that enable the transfer of growth factors and signaling molecules, promoting the formation of organized tissue structures with cellular communication pathways.

Drug Testing and Interaction Studies: The application includes testing drug effects on cell signaling and interaction. The unique feature includes addressable microfluidics that allows controlled diffusion of drugs between wells, enabling the study of drug effects on intercellular signaling and the identification of synergistic or antagonistic drug interactions.

Quorum Sensing in Bacterial Cultures: The application includes studying bacterial communication and biofilm formation. The unique feature includes channels between wells that permit the diffusion of quorum sensing molecules, enabling the study of bacterial communication mechanisms and their role in biofilm development.

Immune Cell Interactions: The application includes investigating communication between different immune cell types. The unique feature includes diffusion-based communication between wells that allows the study of cytokine signaling and immune cell coordination in response to pathogens or cancer cells.

Developmental Biology: The application includes exploring developmental processes and morphogen gradients. The unique feature includes controlled diffusion channels that enable the study of morphogen gradients and their role in developmental patterning and tissue differentiation.

Sensor Networks for Environmental Monitoring: The application includes creating sensor arrays for detecting environmental changes. The unique feature includes diffusive communication between sensor wells that allows the distribution of detected molecules across the network, enhancing the sensitivity and resolution of environmental monitoring systems.

For non-biological applications where a channel can be opened between neighboring addressable wells to establish molecular communication via diffusion in the absence of flow, the following examples are provided: chemical synthesis and reaction networks, catalyst screening, sensor array calibration, material degradation studies, nanomaterial assembly, electrochemical sensing and analysis, crystallization processes, polymer synthesis and characterization, environmental contaminant monitoring, fuel cell research, or the like. These non-biological applications leverage the precise control over molecular diffusion provided by addressable microfluidic systems, enabling advanced research and development in various fields of chemistry, materials science, and environmental monitoring.

Chemical Synthesis and Reaction Networks: The application includes sequential and parallel chemical reactions. The unique feature includes diffusive communication between wells that allows controlled introduction and mixing of reactants, facilitating complex reaction sequences and the study of reaction kinetics.

Catalyst Screening: The application includes high-throughput screening of catalytic materials. The unique feature includes addressable microfluidic wells that enable the diffusion of reactants and products, allowing efficient screening of catalyst performance under various conditions.

Sensor Array Calibration: The application includes calibrating chemical sensors. The unique feature includes controlled diffusion of calibration standards between wells that ensures uniform exposure to different sensor elements, improving calibration accuracy and sensor performance.

Material Degradation Studies: The application includes studying degradation processes of materials. The unique feature includes diffusion channels that allow the controlled exposure of materials to corrosive agents or pollutants, enabling detailed study of degradation mechanisms and rates.

Nanomaterial Assembly: The application includes controlled assembly of nanomaterials. The unique feature includes diffusive transport of nanoparticles between wells that enables precise control over their assembly, facilitating the creation of novel nanostructures and composites.

Electrochemical Sensing and Analysis: The application includes electrochemical detection of analytes. The unique feature includes diffusion-based communication that allows the distribution of electroactive species between electrodes, enhancing the sensitivity and selectivity of electrochemical sensors.

Crystallization Processes: The application includes studying crystallization kinetics and patterns. The unique feature includes controlled diffusion of solutes between wells that enables systematic investigation of crystallization conditions, promoting the development of optimized crystallization protocols.

Polymer Synthesis and Characterization: The application includes creating and analyzing polymer materials. The unique feature includes diffusion channels that permit the gradual introduction of monomers and initiators, allowing controlled polymerization and the study of polymer growth dynamics.

Environmental Contaminant Monitoring: The application includes detecting and analyzing environmental pollutants. The unique feature includes diffusive communication between wells that allows the distribution of environmental samples across sensor arrays, improving detection accuracy and enabling multiplexed analysis.

Fuel Cell Research: The application includes studying fuel cell reactions and performance. The unique feature includes addressable microfluidic systems with diffusion channels that facilitate the controlled delivery of fuel and oxidants, allowing detailed investigation of fuel cell dynamics and efficiency.

Enabling diffusive communication between both horizontal neighbors and in the orthogonal direction in an addressable microfluidic array opens up several unique and advanced applications that go beyond those achievable with only horizontal communication channels. Examples of such applications include, e.g., complex reaction diffusion systems, advances microfabrication and material synthesis, 2D gradient generation for surface chemistry, multidimensional sensor networks, integrated circuit fabrication, controlled drug release systems, 2D cell culture and tissue engineering, high-throughput screening arrays, chemical computing and information processing, dynamic 2D photonic crystals, or the like. These applications leverage the enhanced spatial control and complexity provided by diffusive communication in both horizontal and orthogonal directions, enabling advancements in various fields, from material science and electronics to biotechnology and environmental monitoring.

Complex Reaction-Diffusion Systems: The application includes simulating and studying pattern formation and chemical waves. The unique feature includes orthogonal and horizontal diffusion channels that allow the creation of 2D reaction-diffusion systems, enabling the study of Turing patterns, chemical oscillations, and wave propagation in multiple dimensions.

Advanced Microfabrication and Material Synthesis: The application includes fabrication of complex microstructures and composite materials. The unique feature includes multidirectional diffusion that enables precise spatial control of reactants, facilitating the synthesis of materials with intricate patterns and structures that require control over multiple axes.

2D Gradient Generation for Surface Chemistry: The application includes creating 2D chemical gradients for surface functionalization. The unique feature includes the ability to control diffusion in both horizontal and orthogonal directions that allows the generation of complex 2D gradients, useful for surface chemistry applications such as sensor calibration and the study of surface interactions.

Multidimensional Sensor Networks: The application includes environmental monitoring with high spatial resolution. The unique feature includes diffusive communication in both directions that allows for the creation of a sensor network that can detect and map the distribution of analytes in 2D, enhancing the resolution and accuracy of environmental monitoring systems.

Integrated Circuit Fabrication: The application includes developing advanced integrated circuits and microelectronic devices. The unique feature includes multidirectional diffusion that enables the precise placement and doping of materials, facilitating the fabrication of complex integrated circuits with enhanced performance and miniaturization.

Controlled Drug Release Systems: The application includes development of advanced drug delivery systems. The unique feature includes the ability to control diffusion in 2D that allows the design of drug delivery platforms where the release rate and distribution can be precisely controlled, enabling targeted and sustained drug release.

2D Cell Culture and Tissue Engineering: The application includes creating complex tissue models and studying cell behavior. The unique feature includes multidirectional diffusion that facilitates the establishment of nutrient and chemical gradients in 2D cell culture systems, promoting the development of more physiologically relevant tissue models.

High-Throughput Screening Arrays: The application includes screening chemical reactions, biological assays, or material properties. The unique feature includes the 2D arrangement that allows for high-throughput screening of multiple conditions simultaneously, increasing the efficiency and effectiveness of the screening process.

Chemical Computing and Information Processing: The application includes developing chemical-based computing systems. The unique feature includes multidirectional diffusion channels that enable the implementation of complex logic operations and information processing tasks using chemical reactions, advancing the field of unconventional computing.

Dynamic 2D Photonic Crystals: The application includes developing tunable photonic devices. The unique feature includes control over diffusion in 2D that allows for the dynamic tuning of photonific crystal properties, enabling the development of advanced optical devices with customizable properties.

### Microfluidics Device (400)

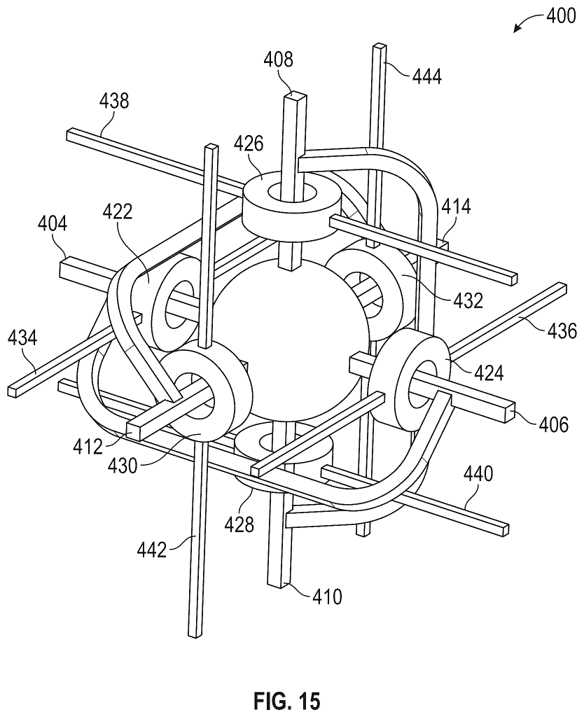

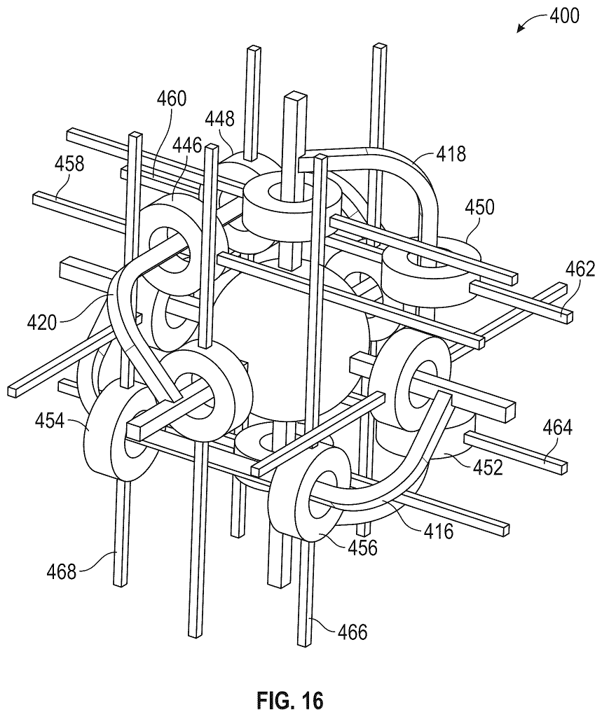

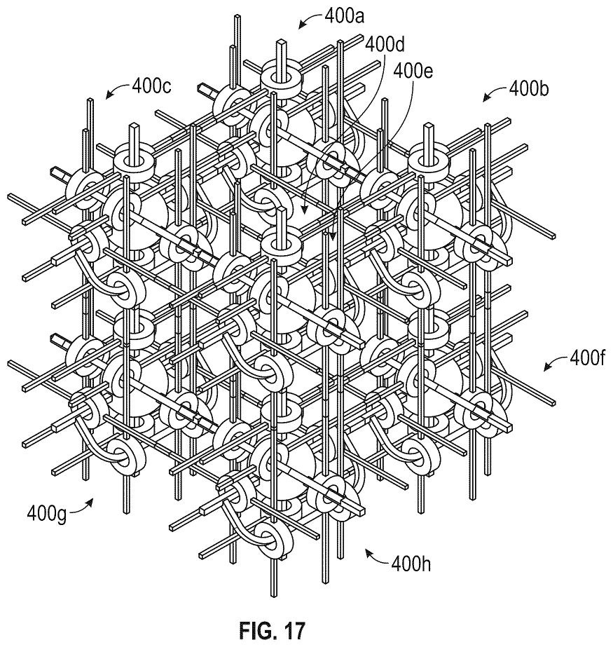

Scaling up the addressable microfluidics plumbing to 3D offers numerous advantages, including enhanced high-throughput experimentation and the ability to conduct more complex experiments. To that end, FIGS. 14-17 show diagrammatic views of an exemplary microfluidic device 400 with row-column-stack addressable plumbing that can be substantially similar in function and/or structure to the other devices discussed, except for the distinctions noted herein. The device 400 defines a 3D flow configuration. The device 400 (a unit module) includes a chamber or well 402 with pairs of input flow channels 404, 406, 408, 410, 412, 414 on opposing sides of the well 402. Bypass flow channel 416 connects channels 404, 406, bypass flow channel 418 connects channels 408, 410, and bypass flow channel 420 connects channels 412, 414, to bypass the well 402 (see FIG. 14). The device 400 of FIG. 15 includes side valves, while the device 400 of FIG. 16 includes both side valves and bypass valves.

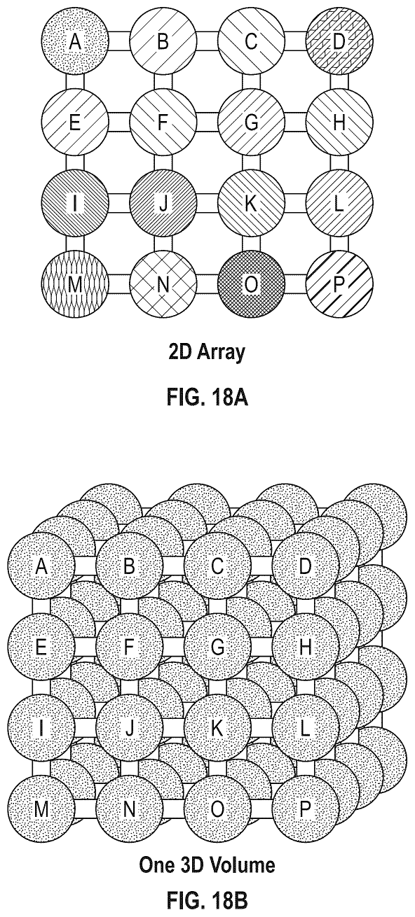

FIG. 15 shows the device 400 including channel-blocking valves 422, 424, 426, 428, 430, 432 (e.g., side valves) for each of the respective flow channels 404, 406, 408, 410, 412, 414, with each of the valves 422, 424, 426, 428, 430, 432 including corresponding lines 434, 436, 438, 440, 442, 444 for actuation of the valves 422, 424, 426, 428, 430, 432. FIG. 16 shows the device 400 including pairs of bypass-blocking valves 446, 448, 450, 452, 454, 456 (e.g., bypass valves) for each of the respective bypass flow channels 416, 418, 420, with each of the valves 446, 448, 450, 452, 454, 456 including corresponding lines 458, 460, 462, 464, 466, 468 for actuation of the valves 446, 448, 450, 452, 454, 456. Although a pair of bypass-blocking valves is shown for the respective bypass flow channels, in some embodiments, a single bypass-blocking valve can be used for the respective bypass flow channels instead. FIG. 17 shows a 2×2×2 array of the devices 400a, 400b, 400c, 400d, 400e, 400f, 400g, 400h connected to each other.

In more detail, FIG. 14 shows a single unit module of the plumbing network for the device 400, with a spherical addressable well 402 (chamber) and three bypasses (channels 416, 418, 420) for routing payloads around the well 402 in the XYZ directions. The bypasses in this configuration divide the 3D space equally to maximize the distance from each other (which helps to overcome fabrication challenges). FIG. 14 omits all valves to demonstrate the “barebones” of the plumbing design.

FIG. 15 adds the flow channel-blocking valves 422, 424, 426, 428, 430, 432 (while still omitting bypass-blocking valves), which makes the device 400 the 3D addressable equivalent of the 2D “Row-Column Addressable Plumbing Layout with Distinct Bypasses” plumbing architecture of device 100 in FIG. 5 and of the 1D “Row-wise Addressable Plumbing Layout with Distinct Bypasses” device 30 in FIG. 3. We term this 3D plumbing layout as: “Row-Column-Stack Addressable Plumbing Layout with Distinct Bypasses” (where “stack” represents the third dimension).

FIG. 16 adds bypass-blocking valves 446, 448, 450, 452, 454, 456 (e.g., bypass valves) to the device of FIG. 15, which makes the device 400 the 3D addressable equivalent of the 2D “Row-Column Addressable Plumbing with Distinct Bypasses and Independent Side Valves” plumbing architecture of device 300 in FIG. 9 and of the 1D “Row-wise Addressable Plumbing with Distinct Bypasses and Independent Side Valves” device 200 in FIG. 8. We term this 3D plumbing layout as: “Row-Column-Stack Addressable Plumbing Layout with Distinct Bypasses and Independent Side Valves”. The device of FIG. 16 can be substantially similar to the device of FIG. 15. However, the device of FIG. 16 includes independent side valves and bypass valves, while the device of FIG. 15 only includes independent side valves.

FIG. 17 stacks the unit modules (devices 400) from FIG. 16 into a 2×2×2 addressable array (size chosen arbitrarily as it could be of any size), to demonstrate the 3D addressable multi-well microfluidics concept.

### Operation Modes

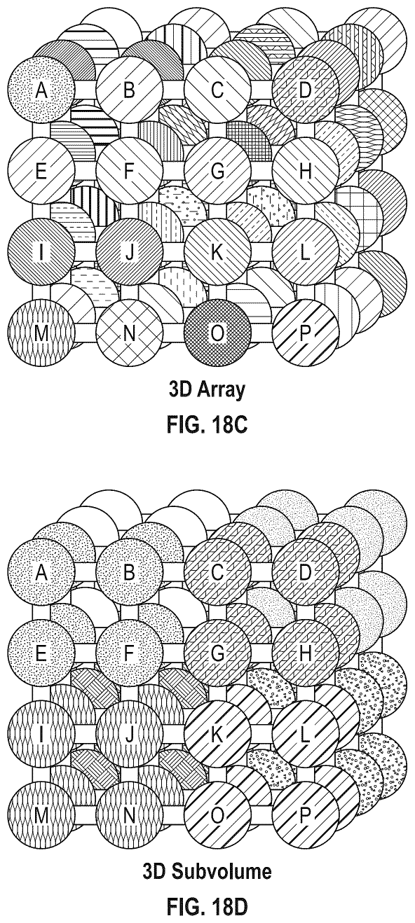

In the context of 3D addressable plumbing, a new mode of operation emerges relative to the conventional array of independent (as indicated by different shades in color) wells shown in FIG. 18A. In this mode, the individual wells are interconnected in all three directions, enabling cells and/or molecular signals to be exchanged between them. This effectively makes the device act as a single 3D experiment or tissue model (see FIG. 18B), but with an ability to deliver and/or sample from any of the lettered locations in the addressable array. A possible application that this technology enables is localized manipulations and observations performed continuously and non-disruptively within thick 3D cultures.

The device still retains the ability to be used as a massively high-throughput screening tool when its wells are isolated (as indicated by different shades in color) from each other (see FIG. 18C). The two modes can be used interchangeably. For example, different cell-drug combinations could be loaded into the individual wells and allowed to be cultured in isolation from each other for an initial period of time. Then, connections between some, or between all, of the wells could be established, thereby giving unprecedented flexibility in terms of the type of time-dependent experiments that can be performed. Likewise, any combination of the two modes can be achieved by breaking the device up into multiple 3D sub-volumes. For example, the 4×4×4 cube in FIG. 18D is broken up into eight 2×2×2 sub-cubes (as indicated by different shades in color; size chosen arbitrarily to illustrate an example). In this setup, the wells belonging to the same sub-cube (i.e., wells with the same shade) can be interconnected with each other, while remaining isolated from the wells in the other sub-volumes. This would achieve four separate 3D experiments being run on the same device. A possible application could be organ-on-a-chip experiments with each sub-volume corresponding to a different organ or tissue model that can be cultures isolated from each other on the same device with communication between the sub-volumes being toggled on or off as desired.

In analogy to the 3D operation modes described above, the 2D addressable plumbing version can also have similar modes of operation: A single 2D plane mode where all wells within the plane are interconnected, enabling continuous diffusion and/or cell migration across the entire plane. A high-throughput screening mode where wells within the 2D array are isolated from each other, allowing independent experiments in each well. A 2D sub-plane mode where the device is divided into multiple 2D sub-planes, each consisting of interconnected wells that are isolated from other sub-planes, enabling multiple independent sub-plane experiments within the same device.

There are several advantages to the device 400 and the arrangement illustrated in FIGS. 18A-18D. Extending addressable microfluidics plumbing to 3D and enabling diffusive communication without flow between neighboring wells in any combination of 3D directions can enable several advanced applications that go beyond the capabilities of 1D and 2D addressable systems. Application of such devices can be (but not limited to), e.g., complex tissue engineering, advanced drug delivery systems, 3D cell culture models, microfabrication of complex devices, high-throughput screening in 3D, dynamic 3D gradient generation, 3D bioprinting, 3D chemical reaction networks, environmental monitoring and remediation, 3D analytical chemistry, 3D computational modeling and simulation, or the like. By extending addressable microfluidics plumbing to 3D and enabling diffusive communication between neighboring wells in any combination of 3D directions, these applications can achieve greater complexity, functionality, and accuracy, opening up new possibilities in research, development, and practical applications across various fields.

Complex Tissue Engineering: The application includes creating fully functional 3D tissue models and organoids. The unique feature includes 3D addressable plumbing that allows precise control of nutrient and oxygen gradients, cell signaling molecules, and scaffold materials in three dimensions, facilitating the growth and differentiation of complex tissues and organ structures.

Advanced Drug Delivery Systems: The application includes developing sophisticated drug delivery platforms. The unique feature includes 3D addressable systems that can create spatially and temporally controlled release profiles for multiple drugs, allowing for more effective and targeted therapeutic strategies, including the study of drug interactions within a 3D environment.

3D Cell Culture Models: The application includes modeling diseases and studying cell behavior in a 3D context. The unique feature includes 3D microfluidics that enables the creation of more physiologically relevant models by allowing cells to interact in a true 3D environment, providing better insights into cellular behaviors and responses compared to 2D or 1D models.

Microfabrication of Complex Devices: The application includes fabricating intricate microfluidic devices and sensors. The unique feature includes 3D addressable microfluidics that allows for the creation of devices with complex geometries and functionalities that cannot be achieved with 2D systems, enabling new types of sensors, actuators, and integrated systems.

High-Throughput Screening in 3D: The application includes performing high-throughput screening of compounds in a 3D matrix. The unique feature includes 3D addressable systems that can screen large libraries of compounds against 3D cell cultures or tissues, providing more accurate data on compound efficacy and toxicity compared to traditional 2D screening methods.

Dynamic 3D Gradient Generation: The application includes studying chemotaxis and other gradient-driven processes. The unique feature includes 3D systems that enable the generation of dynamic chemical gradients in three dimensions, allowing for the study of cell migration, growth, and differentiation in more complex and realistic environments.

3D Bioprinting: The application includes printing complex biological structures. The unique feature includes 3D addressable plumbing that can be integrated with bioprinting techniques to control the deposition of multiple cell types and biomaterials in three dimensions, enabling the fabrication of complex tissues and organs.

3D Chemical Reaction Networks: The application includes exploring reaction-diffusion systems and chemical computing. The unique feature includes 3D addressable systems that allow the study of more complex chemical reaction networks and pattern formation in three dimensions, providing new insights into chemical dynamics and enabling novel chemical computing applications.

Environmental Monitoring and Remediation: The application includes detecting and neutralizing pollutants in a 3D environment. The unique feature includes 3D addressable microfluidics that can create networks of sensors and reactive agents to monitor and remediate environmental pollutants in three dimensions, improving the efficiency and effectiveness of environmental monitoring and cleanup efforts.

3D Analytical Chemistry: The application includes analyzing complex chemical mixtures and reactions. The unique feature includes 3D addressable systems that can spatially separate and analyze components of complex chemical mixtures, providing more detailed and accurate analytical data compared to 2D systems.

3D Computational Modeling and Simulation: The application includes simulating biological and chemical processes in 3D. The unique feature includes 3D addressable microfluidics that can be used to create physical models of complex systems, allowing researchers to simulate and study the interactions and dynamics of these systems in three dimensions.

### Valve Types and Plumbing Improvements

It should be noted that the type of valve utilized in the microfluidic plumbing to block flow channels and bypasses is a free parameter. For example, the plumbing in FIGS. 14-16 utilizes round (e.g., donut-shaped) squeeze valves (shown in FIG. 19A), while the devices in FIGS. 5-6 and 9-13 use square-shaped squeeze valves (shown in FIG. 19B), and the devices in FIGS. 3-4 and 7-8 use variations of the “Quake” valve (shown in FIG. 19C). The valve type choice for these designs was made arbitrarily to signify that different valves can be chosen depending on what is more convenient to fabricate and use. In fact, many other valve types exist and can be used in the exemplary microfluidic devices, e.g., FIG. 19D shows a dome-shaped variation of the Quake valve (see Lee, Y. S. et al., 3D-printed Quake-style microvalves and micropumps, Lab on a Chip, Issue 8 (2018)); FIGS. 19E-G show ribbon, membrane and squeeze valves, respectively (see Noriega, J. L. S. et al., Spatially and optically tailored 3D printing for highly miniaturized and integrated microfluidics, Nature Communications, 12, Article No. 5509 (2021); and Hinnen, H. et al., 3D-Printed Microfluidic One-Way Valves and Pumps, Micromachines, 14, 1286 (2023)); and FIG. 19H is a miniaturized version of the squeeze valve (see Au, A. K. et al., Microvalves and Micropumps for BioMEMS, Micromachines, 2(2), 179-220 (2011)). Therefore, in some embodiments, any of the plumbing designs discussed herein can utilize any of the non-limiting examples of valve types discussed herein, or any combination of them.

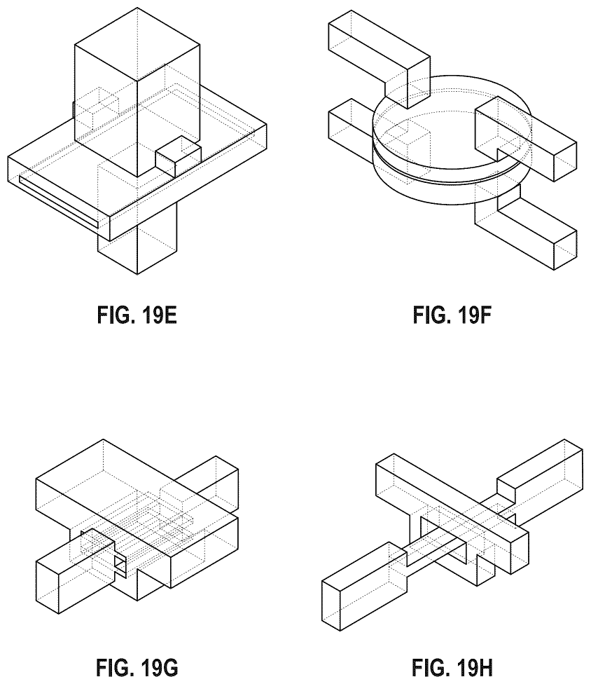

In some embodiments, certain modifications can be made to the valves, and to the plumbing near them, to compensate for various geometric constraints and limitations of the fabrication method. In some instances, it may be difficult to fabricate functional vertically-oriented membranes, because they may be composed of multiple layers 500 fused together (see FIG. 20A). The difficulty can arise when the membrane is inflated and pressure from one side causes the horizontal layers 500 to come apart at the seams at the other side 502 (see FIG. 20B). Specifically, squeezing of a vertical channel is desired, then the squeeze valve would need to be flipped on its side, where the membrane part of the valve would be oriented vertically. In practice, this could be difficult to fabricate using layer-by-layer 3D printing, because there would be “seams” where the layers 500 join together (which would reduce the membrane's functionality). In contrast, a horizontal membrane can be essentially made as a single layer without any seams. In some instances, fabrication methods other than 3D printing could be used. In some instances, 3D printing methods that do not print layer-by-layer could be used.

In some embodiments, a possible solution to the problem illustrated in FIG. 20B can be to introduce a reinforcement 504 whose inter-layer seams are purposely offset from those of the membrane to make delamination less likely (see FIG. 20C). This can be done by either introducing an offset and/or by changing the layer thickness relative to that of the membrane's layers. Alternatively, the reinforcement 504 can be achieved by varying the crosslinking time of the locations adjacent to the membrane and/or the membrane itself (assuming a light-based fabrication method).

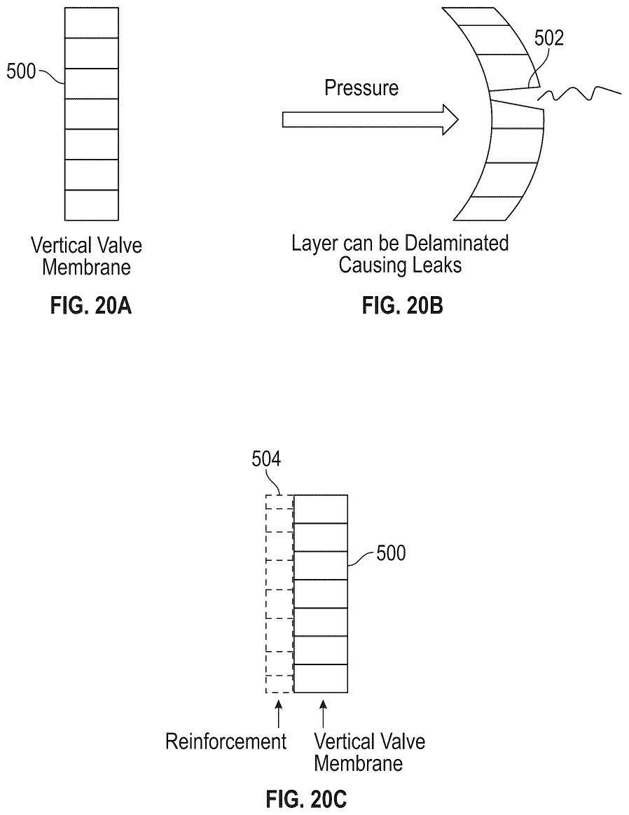

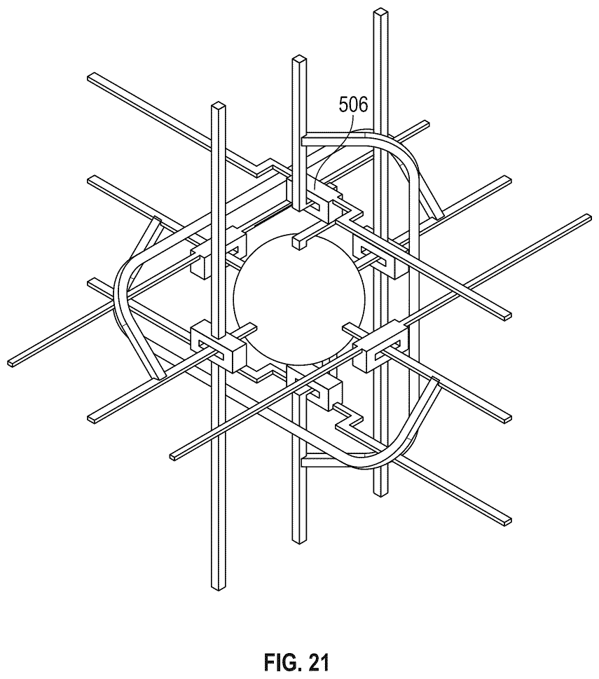

In some embodiments, various changes to the plumbing geometry can be made to compensate for the potential difficulties with vertical valving. (See Hansson, J. et al., Vertical membrane microvalves in PDMS, 28^{th} IEEE International Conference on Micro Electro Mechanical Systems (MEMS) (2015). For example, FIGS. 21 and 22 show the same plumbing design as in FIG. 15, but with the donut-shaped squeeze valves replaced by a miniaturized squeeze valve 506 from FIG. 19H. This exemplary embodiment results in the plumbing elements being further spaced apart from each other, which in turn makes it easier to fabricate the device. Further, FIG. 22 provides a close-up view of a possible plumbing modification that allows maintaining a horizontal squeeze of a vertical flow channel 508, 510 (which is easier to manufacture) through the valve. Specifically, a U-shaped turn 512 is introduced into the vertical flow channel 508, 510 such that a horizontal squeeze valve 506 can block the upper horizontal portion 514 (or lower horizontal portion 516) of the U-shape. The U-shape therefore extends perpendicularly from the vertical flow channel 508, 510 such that the horizontal squeeze valve 506 can be introduced, and the ends of the U-shape connect with respective vertical flow channels 508, 510. The U-shaped turn 512 includes a vertical section 518 extending parallel to the vertical flow channels 508, 510, and connected to opposing ends of the upper and lower horizontal portions 514, 516.

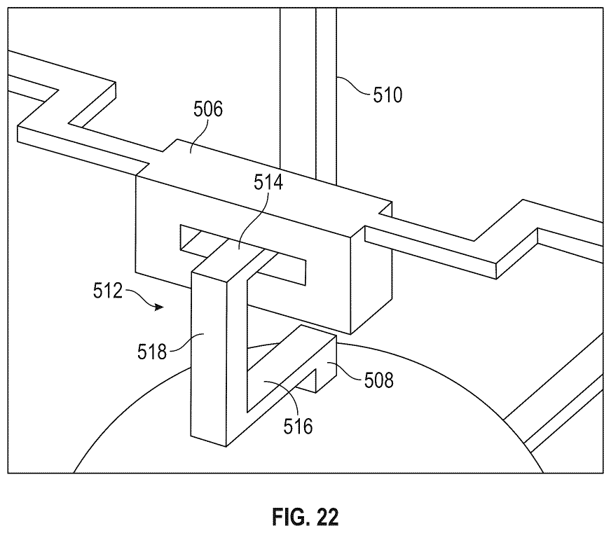

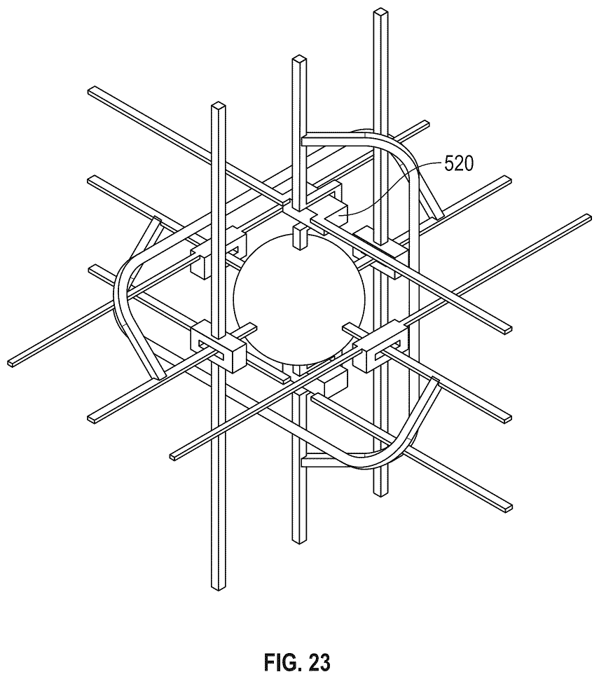

In some embodiments, the design in FIGS. 21 and 22 can be further modified to make the squeezing action more symmetric by placing an inflatable valve 520 at the center of the U-shaped turn 512 that blocks both the upper and lower arms when actuated (see FIGS. 23 and 24). The horizontal sections of the U-shape turn 512 therefore wrap around the valve 520, as illustrated in FIG. 24.

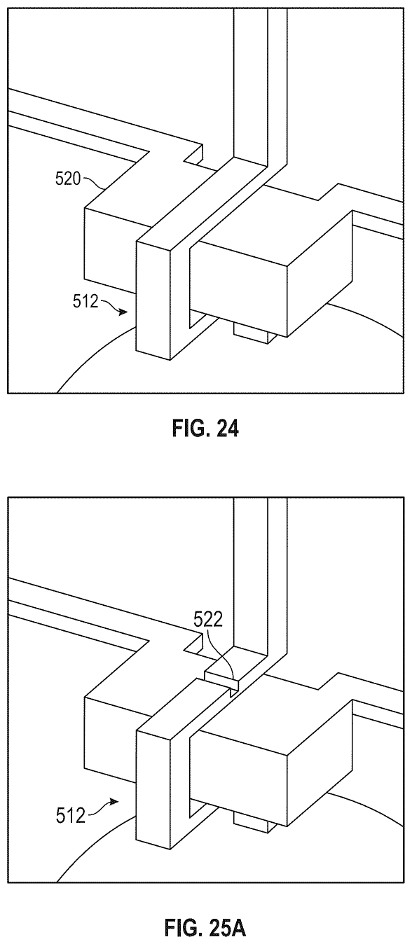

In some embodiments, a modification could be made to the design in FIGS. 23 and 24 by introducing a “doormat” element 522 (i.e., a narrowing in the channel profile) to the squeeze valve to facilitate the blocking of the flow channels. (See, e.g., Lee, Y. S. et al., Weerappuli, P. et al., 3D-printed Quake-style microvalves and micropumps, Lab Chip, 2018, 18, 1207; Novel monolithic “Slightly Open doormat” (SOD) valve enables efficient fabrication of highly-scalable microfluidic gas-on-gas multiplexer, Sensors and Actuators B: Chemical, Vol. 297, 126776 (2019); Hosokawa, K. et al., A normally closed PDMS (polydimethylsiloxane) microvalve, J-Stage, Vol. 120, pages 177-178 (2000); and Au, A. K. et al., Microvalves and Micropumps for BioMEMS, Micromachines, 2(2), 179-220 (2011)). This is demonstrated in FIGS. 25A and 25B. In particular, the horizontal sections of the U-shape turn 512 include narrowed areas defined by grooves, elevations, channels, discs, hemispheres or other shapes formed therein that form the doormat element 522 disposed on one or both sides of the valve.

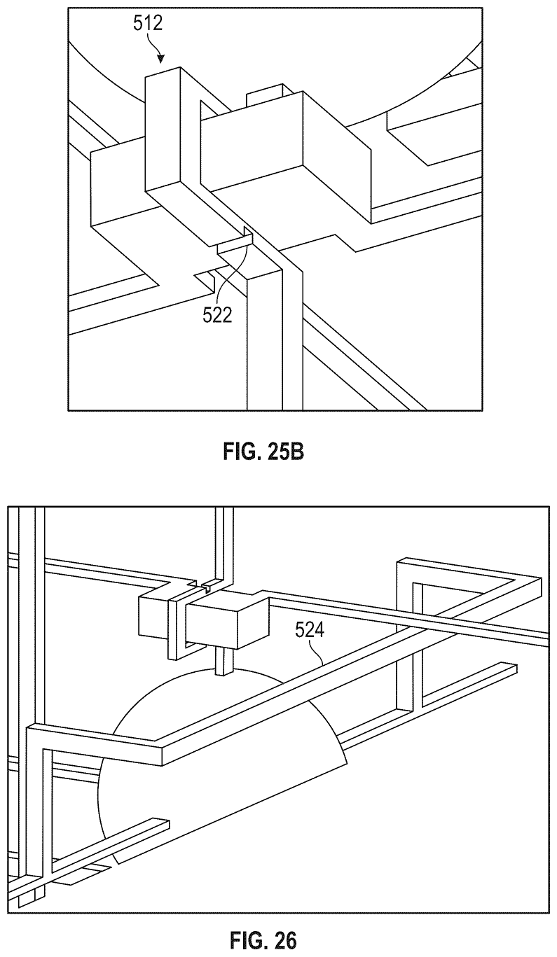

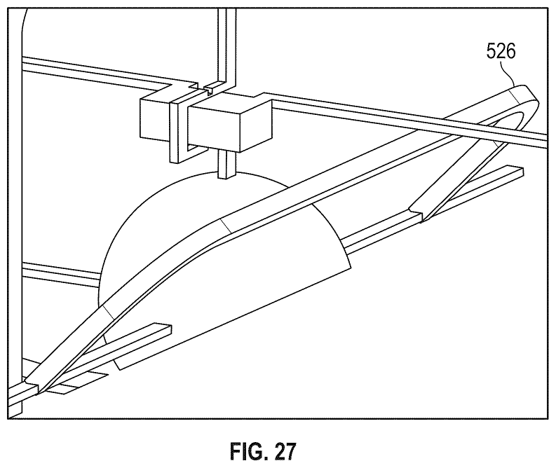

In some embodiments, the bypass could consist of only straight horizontal and vertical channel segments 524 with right angle turns (see FIG. 26) to facilitate the fabrication process, as it might be challenging to create diagonal bypasses and curved features 526 (see FIG. 27) due to technological limitations. For example, layer-by-layer 3D printers can make straight features better than diagonal or curved ones. Likewise, the rounded corners in the same figure may pose a fabrication challenge as well. However, any of the designs could be used to achieve the desired structure and functionality of the microfluidic device.

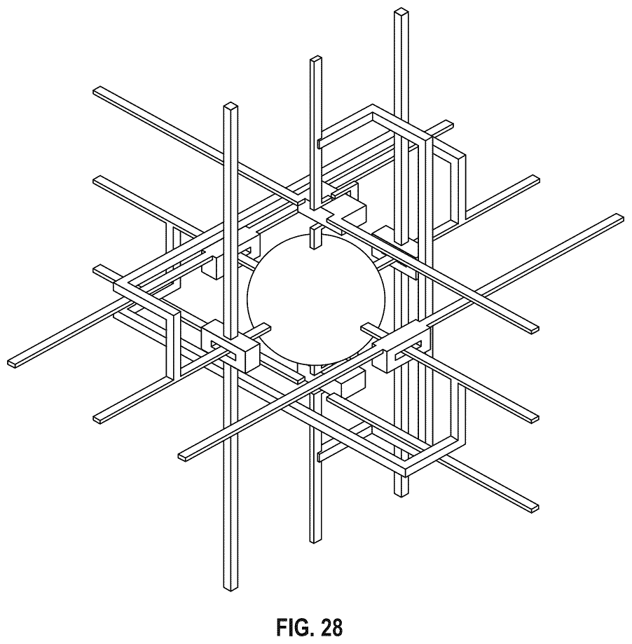

To that end, FIG. 28 demonstrates what a unit module with such straight segment microfluidic plumbing may look like. It can be seen that the design does not contain any diagonal elements or rounded corners, thereby making it potentially easier to fabricate with methods including (but not limited) to 3D printing.

Overall, FIGS. 21-28 demonstrate the large variety of valve choices and plumbing modifications available for the exemplary microfluidic device design optimization to overcome fabrication difficulties. Therefore, in some embodiments of the microfluidic technology disclosed herein, different valve types (and combinations of valve types) and associated plumbing modifications may be used.

### Materials

In some embodiments, the microfluidic devices discussed herein can be fabricated using any known method and material. Therefore, the examples provided herein are non-limiting. In some embodiments, the devices discussed herein can be fabricated from a material and/or method that results in a device having flexible parts for valves and has solid parts for the body. As discussed herein, in some embodiments, the devices discussed herein can be fabricated using 3D printing to either generate the device either in parts or as a one-piece component (in which case different settings may be used to generate the flexible parts of the device vs the solid ones). Furthermore, in some embodiments the material could be crosslinkable in case a light-based fabrication method, such as Digital Light Processing (DLP) or Stereolithography (SLA) 3D printing is used. However, it should be understood that the devices are not limited to 3D printing fabrication methods; nor do their materials have to be crosslinkable (e.g., in case a non-light-based fabrication method is used). Given that the entire device can be fabricated in one process, the device can be made from a material that is flexible enough to support the on-chip membranes required by the valves of the addressable plumbing design. The material that has been used for fabricating traditional microfluidics devices with valves by, e.g., bonding together of 2D layers each of which is prepared via soft lithography, generally includes poly(dimethyl siloxane) (PDMS), which has the following mechanical properties: an elastic modulus of 1.32-2.97 MPa and an elongation at break (i.e., stretchability) of ˜40%. (See, e.g., Tong, A., et al., A minireview of microfluidic scaffold materials in tissue engineering. Frontiers in Molecular Biosciences, 8: p. 783268 (2022)). Therefore, the material used for the fabrication of the devices disclosed herein could in some embodiments potentially have similar mechanical properties to make functional microfluidic valves needed for the localized fluid and cell manipulations within the device. (See, e.g., Tong, A., et al., Automated Addressable Microfluidic Device for Minimally Disruptive Manipulation of Cells and Fluids within Living Cultures. ACS Biomaterials Science & Engineering, 6(3): p. 1809-1820 (2020)).

Furthermore, in some embodiments, the microfluidic technology disclosed herein may be used for culturing living organisms, such as: cells, bacteria, fungi, viruses, etc. In this case, material should be biocompatible and/or optionally transparent in case imaging is desired. In case of adherent organisms, the material should be made to support the attachment of such organisms. Moreover, in some embodiments the technology may be used for a tissue engineering scaffold, in which case the material should be made biodegradable: either with a tunable degradation time (so that the growth of the natural material can displace the artificial one) or that can be digested by a chemical flowed through its channels. Overall, it should be understood that the current disclosure is not tied to any one fabrication methodology/technology or application; and the discussion herein is meant to anticipate any possible modifications to the microfluidic plumbing and the source materials used to make it in the face of difficulties generating the envisioned micro-architectures and applications with additional requirements.

While exemplary embodiments have been described herein, it is expressly noted that these embodiments should not be construed as limiting, but rather that additions and modifications to what is expressly described herein also are included within the scope of the invention. Moreover, it is to be understood that the features of the various embodiments described herein are not mutually exclusive and can exist in various combinations and permutations, even if such combinations or permutations are not made explicit herein, without departing from the spirit and scope of the invention.

1. An addressable microfluidics device, comprising:
a chamber;
a first flow channel pair including a first flow channel and a second flow channel fluidly connected to sides of the chamber;
a second flow channel pair including a third flow channel and a fourth flow channel fluidly connected to sides of the chamber;
at least one bypass flow channel connecting either (i) the first flow channel and the second flow channel, or (ii) the third flow channel and the fourth flow channel, to bypass the chamber; and
a first side valve associated with either the first flow channel or the third flow channel, and a second side valve associated with either the second flow channel or the fourth flow channel, wherein the first and second side valves are selectively operable into open or closed positions to divert flow through the at least one bypass flow channel.

2. The addressable microfluidics device of claim 1, wherein the chamber is capable of (i) receiving and/or sending a fluid (ii) receiving and/or sending the fluid with a sample, (iii) receiving and/or sending the fluid with a chemical signal, and/or (ii) hosting a reaction and/or a culture.

3. The addressable microfluidics device of claim 1, wherein the first and second flow channels extend along a first plane.

4. The addressable microfluidics device of claim 3, wherein the third and fourth flow channels extend along a second plane offset from the first plane.

5. The addressable microfluidics device of claim 4, wherein the second plane is offset from the first plane by 90°.

6. The addressable microfluidics device of claim 1, wherein the at least one bypass flow channel includes a first bypass flow channel connecting the first flow channel and the second flow channel, and a second bypass flow channel connecting the third flow channel and the fourth flow channel, to bypass the chamber.

7. The addressable microfluidics device of claim 6, wherein the first side valve is associated with the first flow channel and the second side valve is associated with the second flow channel, and wherein the first and second side valves are selectively operable into the open or closed positions to divert flow through the first bypass flow channel.

8. The addressable microfluidics device of claim 7, comprising a third side valve associated with the third flow channel and a fourth side valve associated with the fourth flow channel, wherein the third and fourth side valves are selectively operable into open and closed positions to divert flow through the second bypass flow channel.

9. The addressable microfluidics device of claim 1, wherein the first side valve and the second side valve operate concurrently to open or close in a joined manner.

10. The addressable microfluidics device of claim 1, wherein the first side valve and the second side valve are independently operable relative to each other to open and close.

11. The addressable microfluidics device of claim 1, comprising at least one bypass valve associated with the at least one bypass flow channel, wherein the at least one bypass valve is operable into open or closed positions to divert flow through the at least one bypass flow channel.

12. The addressable microfluidics device of claim 8, comprising a fifth flow channel and a sixth flow channel fluidly connected to sides of the chamber.

13. The addressable microfluidics device of claim 12, wherein the first and second flow channels extend along a first plane, the third and flow channels extend along a second plane offset from the first plane, and the fifth and sixth flow channels extend along a third plane offset from both the first plane and the second plane.

14. The addressable microfluidics device of claim 13, wherein the at least one bypass flow channel includes a third bypass flow channel connecting the fifth flow channel and the sixth flow channel.

15. The addressable microfluidics device of claim 14, comprising a fifth side valve associated with the fifth flow channel and a sixth side valve associated with the sixth flow channel, wherein the fifth and sixth side valves are selectively operable into open or closed positions to divert flow through the third bypass flow channel.

16. A method for microfluidics operation, comprising:
passing a fluid through at least one of (i) a first flow channel fluidly connected to a chamber of an addressable microfluidics device, (ii) a second flow channel fluidly connected to the chamber, (iii) a third flow channel fluidly connected to the chamber, (iv) or a fourth flow channel fluidly connected to the chamber, wherein:
the first flow channel and the second flow channel form a first flow channel pair connected to sides of the chamber; and
a third flow channel and a fourth flow channel form a second flow channel pair connected to sides of the chamber;
selectively operating a first side valve associated with either the first flow channel or the third flow channel, and/or selectively operating a second side valve associated with either the second flow channel or the fourth flow channel, into open or closed positions to divert flow through at least one bypass flow channel connecting either (i) the first flow channel and the second flow channel, or (ii) the third flow channel and the fourth flow channel, to bypass the chamber.

17. An addressable microfluidics device, comprising:
a chamber;
a first flow channel fluidly connected to a first side of the chamber;
a second flow channel fluidly connected to a second side of the chamber;
a bypass flow channel connecting the first flow channel and the second flow channel;
a first side valve associated with the first flow channel; and
a second side valve associated with the second flow channel;
wherein the first and second side valves are selectively operable into open and closed positions to divert flow through the bypass flow channel.

18. The addressable microfluidics device of claim 17, wherein the chamber is capable of (i) receiving and/or sending a fluid (ii) receiving and/or sending the fluid with a sample, (iii) receiving and/or sending the fluid with a chemical signal, and/or (ii) hosting a reaction and/or a culture.

19. The addressable microfluidics device of claim 17, wherein the first side valve and the second side valve are independently operable relative to each other to open and close.

20. The addressable microfluidics device of claim 17, comprising at least one bypass valve associated with the bypass flow channel, wherein the at least one bypass valve is operable into open or closed positions to divert flow through the bypass flow channel.
